(19) United States
(12) Patent Application Publication
(10) Pub. No.: US 2010/0057401 A1
(43) Pub. Date: Mar. 4, 2010

(54) SYSTEM, METHOD AND KIT FOR TESTING GAS MONITORS

(76) Inventors: Arthur Scheffler, Surrey (CA); Cristian D. Nanea, Vancouver (CA)

Correspondence Address: 3M INNOVATIVE PROPERTIES COMPANY, PO BOX 33427, ST. PAUL, MN, 55133-3427, US

(21) Appl. No.: 12/530,682

(22) PCT Filed: Mar. 20, 2008
(86) PCT No.: PCT/US08/57573

§ 371 Date: Sep. 10, 2009

Related U.S. Application Data
(60) Provisional application No. 60/999,747, filed on Apr. 2, 2007.

(51) Int. Cl. G06F 15/00; G01N 33/00

(52) U.S. Cl. 702/182; 73/1.06

(57) ABSTRACT

System and method are utilized for testing the performance of a gas monitor against predetermined monitor characteristics to determine if performance of a gas monitor is validated following testing gas being directly delivered to a gas sensor of the gas monitor.

### BACKGROUND

The present disclosure relates to gas testing processes and systems and, more particularly, to testing methods and systems for validating performance of gas monitors, such as carbon monoxide monitors.

A variety of toxic gases are monitored for dangerous concentrations. One such gas is carbon monoxide, (CO), a colorless, tasteless, odorless, and deadly gas. CO in high concentrations is not only undetectable by humans but is also highly dangerous and widely prevalent in many everyday situations. For instance, carbon monoxide can be produced by combustion of a number of common household sources, including wood or gas fireplaces, gas or oil furnaces, wood stoves, gas appliances, etc. CO typically becomes unsafe when dangerous concentrations build-up due to, for example, poor ventilation. CO build-up is a potential problem, for example, in energy-efficient, airtight homes, vehicles, and plants that decrease the exchange of inside and outside air.

CO monitors are commonly used to determine if the level of CO gas in the air has become dangerous. These devices continuously monitor the air for impermissible CO concentrations. The monitors calculate whether CO levels are high enough to pose a risk of dangerous buildups in the human body. If CO levels become so high, the monitors will issue an alarm.

To ensure adequate environmental monitoring, CO monitors are routinely checked to confirm their reliability. Prior attempts to provide performance validation typically occur after a monitor is manufactured and again after the monitor has been installed. Known validation protocols require that the monitors be tested over generally prolonged testing periods.

Known testing procedures generally require lengthy testing times because the sensor must reach an equilibrium response to the test gas before testing can proceed. Some testing procedures may take 10-15 minutes, while others may take up to 4 hours, depending on the nature of the monitor's specifications. For example, a gas sensor may be validated if a reading of the sensor (a) occurs within a time (usually several minutes or longer) based on the sensor reaching greater than 90% of its equilibrium response; and, (b) falls within an acceptable range of values based on the concentration of testing gas being used. Since testing procedures use testing gas, and given the relatively lengthy times required for validating a monitor's performance, considerable testing gas may be used. It will be appreciated that there are cost considerations when frequently using relatively expensive testing gases for the significant periods of time as noted above, especially when such costs are multiplied by the number of sensors to be monitored and the number of times the monitors will be tested. If the testing gas is toxic, undesirable safety issues may also be present, should the gas not be handled properly or the testing procedure not be properly carried out.

As noted, some known testing procedures apply a testing gas to the detector. Some known procedures may simulate conditions in which an alarm signal would issue a warning when exposed to undesirable levels of such a gas. Typically, such testing is performed by applying the test gas from a gas canister to a region or space exterior of the gas monitor's housing. Generally, considerable care is exercised in order to insure proper delivery of the testing gas in a safe manner. In one specific example, a gas impervious plastic bag surrounds the gas monitor for confining the gas during testing. A gas delivery tube has one end connected to a gas regulator associated with a testing gas canister and a gas delivery end connected to the plastic bag. The gas delivery tube end and plastic bag are placed exterior of and in close proximity to the gas monitor during the testing. The same user also opens the regulator and applies the testing gas. The user must wait for a specified time for insuring that the test protocol is followed. Typically, for such a gas monitor to pass a test, an alarm should sound within period of about 10-15 minutes. This is a considerable amount of time to expend not only in terms of holding the delivery tube and plastic bag in proper position over the gas monitor, but also for using the relatively expensive testing gas. This approach also tends to increase the time to validate a gas monitor because the applied testing gas must purge the volume of air surrounding the gas sensor, whereby the sensor can react to a constant level of testing gas at the desired level of testing gas concentration. Accordingly, not only is the amount of actual testing time at the desired level of testing gas concentration relatively lengthy, but the actual time to set-up and perform a test is increased due to additional time delays arising from setting up the test and purging the air.

One significant improvement is described in commonly-assigned and copending U.S. patent application having U.S. Ser. No. 11/551,828 filed in the U.S. Patent and Trademark Office on Oct. 23, 2006. In the described approach, validations of gas sensors of gas monitors are determined through a process involving direct application of testing gas coupled with a quick determination of a sensor's response through a testing mechanism. In particular, use is made of a testing device fixed with the gas monitor that relies upon use of an algorithm for determining the validity of gas monitor performance in a quick and reliable manner. While such an approach is highly successful, nonetheless efforts are being undertaken for continuing generation of improvements in this field that are efficient and economical.

### SUMMARY

In one exemplary implementation, the present disclosure is directed to a method adapted for testing a gas sensor assembly, the method comprises: coupling a portable tool to the gas sensor assembly; receiving test data representative of performances of the gas sensor assembly in response to testing gas by a data receiving device on the portable tool; and determining performance of the gas sensor assembly based on processing the received test data.

In another exemplary implementation, the present disclosure is directed to a gas testing system adapted for use in testing a gas sensor assembly, the system comprising: a portable tool; a data receiving device on the portable tool; a data processing system on the portable tool and including a testing module; the data processing system is operable for receiving test data relating to testing gas values sensed by a gas sensor assembly, wherein the testing module is operable for determining performance of the gas sensor assembly based on the received test data.

In an illustrated embodiment, provision is made for a gas monitor testing kit comprising: a portable gas testing system for testing a gas sensor assembly; and a fluid coupling apparatus for delivering testing gas to a gas sensor assembly of a gas monitor assembly.

These and other features and aspects of this disclosure will be more fully understood from the following detailed description of the preferred embodiments. It should be understood that the foregoing generalized description and the following detailed description are exemplary and are not restrictive of the disclosure.

### Glossary

The term “equilibrium response” as used in the specification and claims defines a response when the sensor output of the gas sensor of the gas monitor apparatus being tested no longer increases.

The term “wireless” as used in the specification and claims defines any type of electrical or electronic operation which is accomplished without the use of a so-called hard wired or physical connection. The term is normally used in the telecommunications industry to refer to systems (e.g., radio transmitters and receivers, remote controls, computer networks, each use some form of energy radio frequency (RF), infrared light, laser light, acoustic energy, and microwave energy) without the use of wires or conductors.

### BRIEF DESCRIPTION OF THE DRAWINGS

FIG. 1 is a schematic view of a gas monitoring system that includes a field test kit.

FIG. 2 is a perspective view of a gas monitor apparatus of a gas monitoring system.

FIG. 3 is a side view of the gas monitor apparatus illustrated in FIG. 2.

FIG. 4 is an exploded perspective view of the gas monitor apparatus illustrated in FIGS. 2 and 3.

FIG. 5A is a front view of a fluid coupler apparatus usable with the present disclosure.

FIG. 5B is a rear view of the fluid coupler apparatus shown in FIG. 5A.

FIG. 5C is an enlarged cross-sectional view of a part of the fluid coupler apparatus illustrated in FIGS. 5A & 5B.

FIG. 6 is a right side view of the fluid coupler apparatus illustrated in FIG. 5.

FIG. 7 is a view of the fluid coupler in a coupled condition relative to an electronic control assembly of the gas monitor.

FIG. 8 is a graph illustrating response curves of gas sensor assemblies that may be utilized in the gas monitor apparatus depicted in FIGS. 2 and 3

FIG. 9 is a simplified block diagram illustrating an electronic control assembly of the gas monitor.

FIG. 10 is a flow diagram illustrating one aspect of an improved testing process of this disclosure wherein a digital processor is mounted within the gas monitor apparatus.

FIG. 11 is a flow diagram illustrating another aspect of an improved testing process of this disclosure.

FIG. 12 is a graph illustrating response curves of gas sensor assemblies that may be utilized in this disclosure.

FIG. 13 is a perspective view of the portable testing device spaced from a gas monitor, as well as a testing gas fluid coupler spaced from the gas monitor prior to their installation to the gas monitor.

FIG. 14 is a perspective view of the portable testing device physically coupled to a gas monitor.

FIG. 15 is an exploded perspective view of a portable testing device that is to be coupled to a gas sensor assembly of a gas monitor for testing performance of the later.

FIG. 16 is a simplified block diagram of an electronic control assembly employing aspects of the present disclosure.

FIGS. 17A & 17B represent a flow diagram illustrating another aspect of an improved testing process of this disclosure.

FIG. 18 is schematic diagram of a wireless testing tool including aspects of the present disclosure.

FIG. 19 is a simplified block diagram of a network including aspects of the present disclosure.

### DETAILED DESCRIPTION

The words “a,” “an,” and “the” are used interchangeably with “at least one” to mean one or more of the elements being described. By using words of orientation, such as “top,” “bottom,” “overlying,” “front,” “back” and “backing” and the like for the location of various elements in the disclosed articles, we refer to the relative position of an element with respect to a horizontally-disposed body portion. We do not intend that the disclosed articles should have any particular orientation in space during or after their manufacture.

The present disclosure improves upon known testing methods and systems for validating performances of gas monitors. In so doing, it addresses needs for validating gas monitor performance quickly and reliably and yet simply and efficiently.

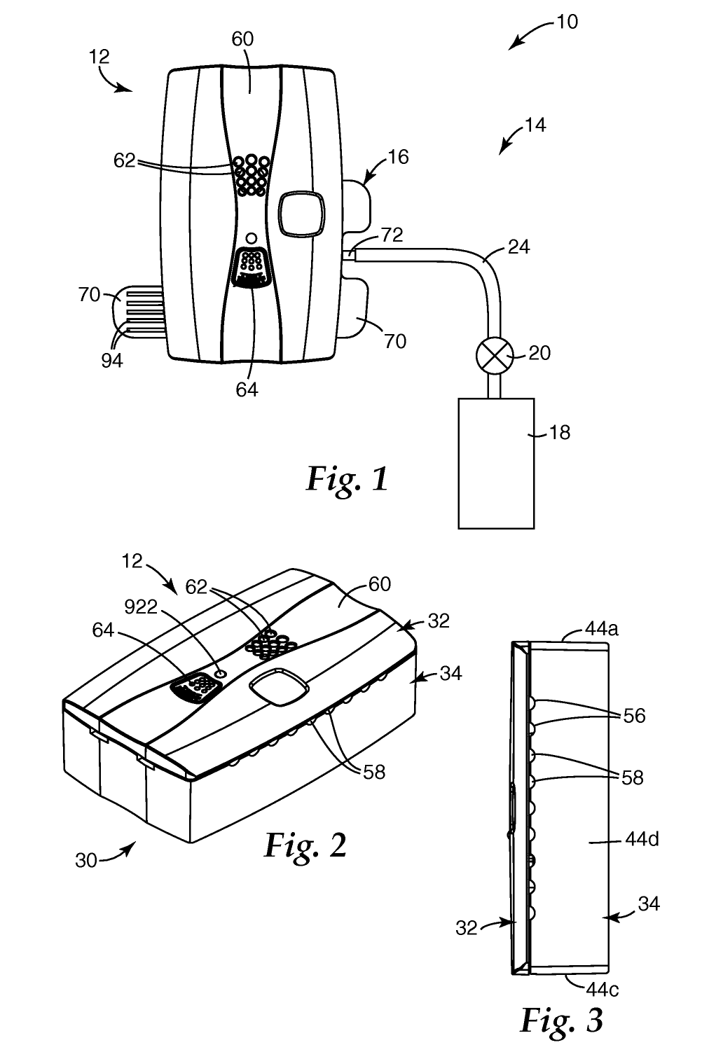

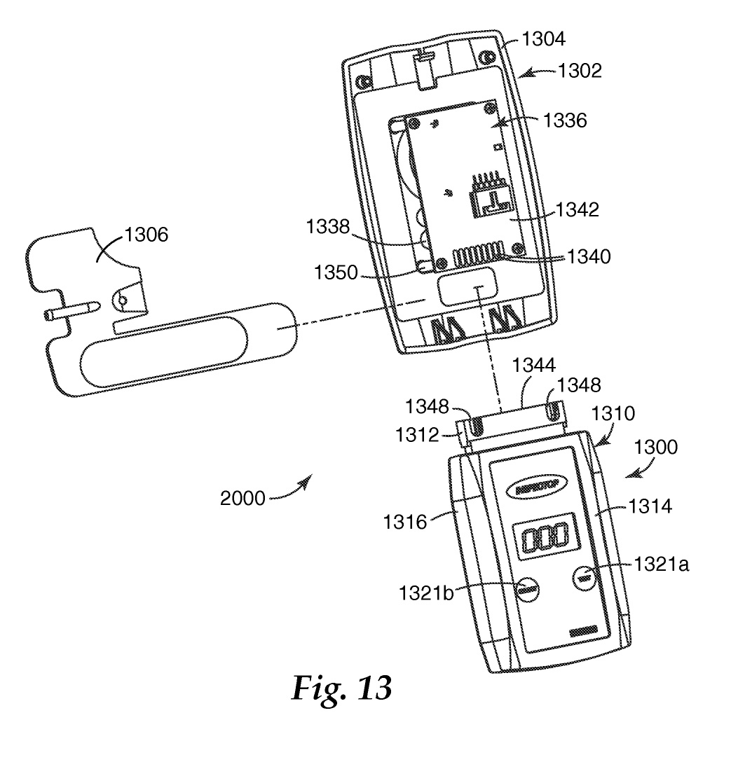

FIGS. 1-12 illustrate and describe a gas monitoring system and method as set forth in applicants' copending and commonly assigned U.S. Patent Application having Ser. No. 11/551,828 filed on Oct. 23, 2006 which is incorporated herein and made a part hereof. FIGS. 1-12 are related to a gas sensor testing algorithm that resides in the gas monitor. FIGS. 13-19 illustrate and describe aspects of the presently claimed invention that relate to a monitoring system and method, that perform gas monitoring using a portable and/or networked arrangement remote from a gas monitor. Accordingly, aspects of the present disclosure described in the previously noted patent application (FIGS. 1-12) that are relevant to a description of the present disclosure as described FIGS. 13-19 have been set forth.

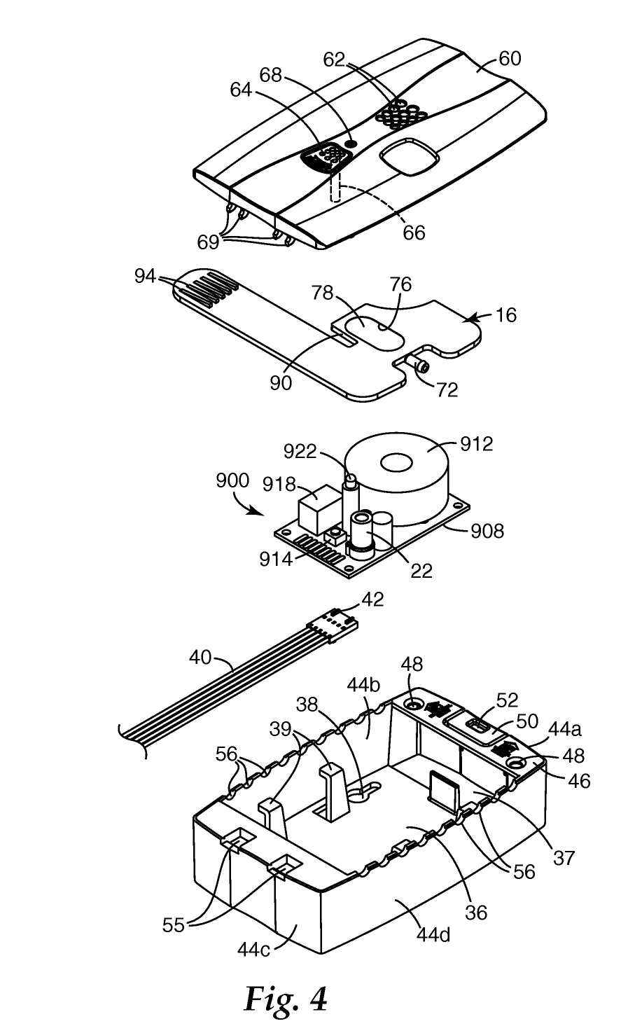

FIG. 1 is a schematic view of a gas monitoring system 10 operable for confirming performance of a carbon monoxide gas monitor apparatus 12. Included in the gas monitoring system 10 is a field test kit assembly 14. The field test kit assembly 14 includes a fluid coupling apparatus 16 also made according to this disclosure. The fluid coupling apparatus 16 is adapted to couple a source of testing gas, such as from a testing gas canister 18 that flows through a regulator 20, to a gas sensor assembly 22 (FIG. 4) within in the gas monitor apparatus 12 by way of flexible tubing 24. While the illustrated embodiment is described in the context of a carbon monoxide gas monitor apparatus 12, this disclosure is broadly capable of validating performances of not only other kinds of CO gas monitors, but other gas monitors for other gases as well. This testing determines whether the gas monitor apparatus satisfies its performance criteria without the gas monitor apparatus having to run a complete test. Basically, the testing is accomplished in durations much shorter than the normal testing periods for CO gas monitors. Accordingly, the shorter testing periods produce, significant savings since less testing gas is consumed than otherwise, and the attendant testing labor costs are reduced.

The gas monitor apparatus 12 is adapted for operation in home or commercial environments although it may be operated in a variety of other environments. As illustrated in FIGS. 1-4, the gas monitor apparatus 12 may have a generally parallelepiped enclosure or housing assembly 30. The housing assembly 30 may be made of any suitable materials, such as a thermoplastic material, for example, polycarbonate, ABS or the like. The housing assembly 30 can have a variety of configurations and includes essentially a front cover assembly 32 removably attached to a back plate assembly 34. The back plate assembly 34 includes an intermediate flat back wall 36 which defines openings 37 at opposite ends thereof (only one of which is shown in FIG. 4). The back wall 36 has suitable apertures 38(one of which is shown) that facilitate attachment to any suitable supporting structure (not shown). The back wall 36 may have other configurations and be structured differently for enabling the attaching thereof to other kinds of supporting structures. For example, the back wall 36 may have suitable structure (not shown) for allowing releasable attachment to an electric box (not shown), such as when the gas monitor apparatus 12 is to be hardwired. Also, the back wall 36 may have other structure, such as projections 39 for allowing routing of a wiring harness 40 (FIG. 4) attached to a connector 42. The connector 42 is attached to the electronic control assembly. The openings 37 allow the wiring to extend out of the gas monitor 12 for coupling to a power source. Other suitable housing construction for battery powered or main powered systems are envisioned.

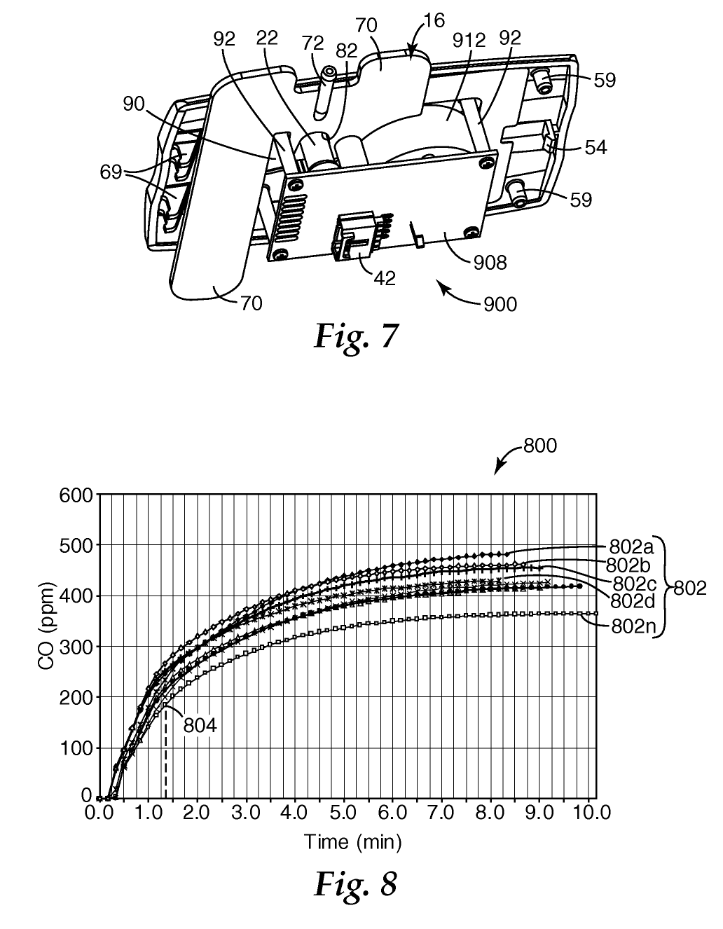

The sidewalls 44a-44d extend upwardly relative to the back wall 36 as viewed in FIG. 4 The top sidewall 44a includes an overhang portion 46 that includes a pair of spaced apart openings 48. A user-depressible finger latch 50 is integrally formed into the sidewall 44a. The finger latch 50 has a latch opening 52 in a distal portion that lies within the overhang thereof for releasable cooperation with a tab 54 (FIG. 7) extending laterally from an inner wall of the front cover assembly 32. The finger latch 50 is normally biased to latch with the tab 54 to retain the former to the latter. A pair of spaced apart openings 55 is in the bottom sidewall 44c for cooperating with the front cover assembly 32.

As illustrated in FIG. 4, the sidewalls 44b and 44d have a series of scalloped portions 56 along their edges, such that when they mate with a surface of the front cover assembly 32 they define a series of lateral openings 58 (FIGS. 2 & 3). The lateral openings 58 allow for ambient air to travel into and through the interior of the gas monitor apparatus 12 for sensing purposes. A pair of spaced apart projections 59 (FIG. 7) is adapted to cooperate with the openings 48 on the back plate assembly so as to assist in properly mating the latter to the front cover assembly, whereby the front cover assembly can pivot relative to the back plate assembly between open and closed conditions. While the present embodiment discloses the foregoing such structure for effecting pivoting, other approaches for pivotally or otherwise opening the front cover assembly 32 of the gas monitor apparatus 12 are envisioned.

The front cover assembly 32 has a generally rectangular shape panel portion 60 formed with a series of openings 62 that facilitate passage of air and sound therethrough. The front cover assembly 32 also includes a finger actuated switch element 64 depressed by a user from its normally non-operative state to an operative state or testing mode for actuating a gas testing process in accordance with this embodiment. In this embodiment, the finger actuated switch element 64 includes an actuator rod 66 (FIG. 4) connected to an underneath portion of the switch element 64 and is adapted to engage a switch as will be described. In addition, a display opening 68 is provided, whereby a display, to be described, can protrude for display purposes. In addition, a pair of spaced apart curved legs 69 (FIG. 4) is normally adapted to be positioned within the openings 55 and cooperates with the back plate assembly for allowing the front cover and back plate assemblies 32, 34; respectively, to be generally pivotally moved, as in a clam-shell fashion, between a closed condition (FIG. 2) and an open position (not shown) as is known. Contemplated is a variety of other suitable approaches for releasably joining the two assemblies together.

### Fluid Coupling Apparatus of Field Test Kit

In FIGS. 4-7, the fluid coupling apparatus 16 is seen as being constructed to allow delivery of testing gas to the gas monitor apparatus 12 in an easy and inexpensive fashion. As such, this allows field testing to be more easily accomplished. In particular, the fluid coupling apparatus 16 is removably couplable to the gas monitor and delivers the testing gas to a region positioned immediately adjacent a gas sensor assembly, thereby making for a more efficient testing process as will be explained. The regulator 20 (FIG. 1) is controlled by the user for controlling the testing gas admitted into the tubing 24 and that flows to the gas monitor apparatus 12.

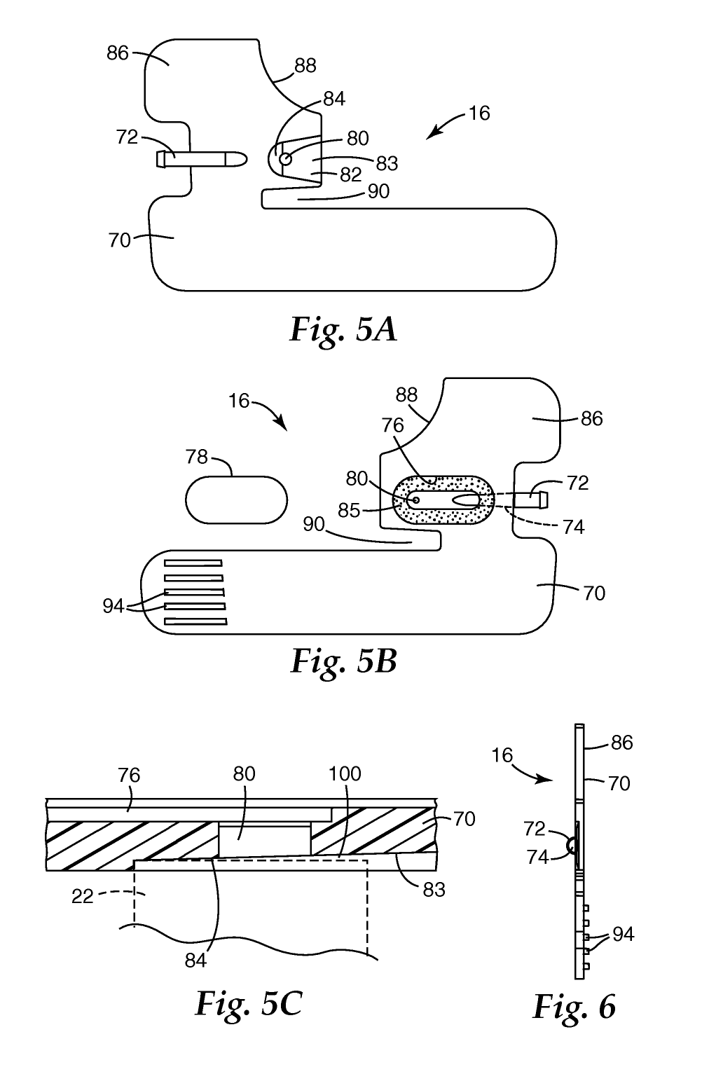

The fluid coupling apparatus 16 may be defined by an elongated and thin fluid coupler body 70 that may be made of a suitable thermoplastic material, such as nylon, polycarbonate, ABS or the like. Other suitable materials and constructions of the housing assembly are contemplated. The tubing is releasably coupled to a tube barb 72 protruding generally longitudinally therefrom so as to be exteriorly located when the fluid coupling apparatus is in the testing mode. An internal passageway 74 (FIGS. 5A, 5B, 5C & 7) is formed in the fluid coupler body 70 and extends through the tube barb 72 and terminates in a laterally disposed recess 76 (FIG. 5B) formed intermediate the length of the fluid coupler body 70. While a fluid passageway is formed internally, it is also envisioned that the fluid passageway may be external to the fluid coupler body 70.

The fluid coupler body 70 is also provided with a gas sealing member 78 that serves to cover one portion of the recess 76 to provide a gas seal. The gas sealing member 78 may be a thin plastic or the like that covers the recess 76 in a flush manner to provide the gas seal. The recess 76 has an enlarged mouth portion into which the testing gas enters as it exits the passageway 74.

Reference is made to FIG. 5B for illustrating a gas delivery opening 80 in fluid communication with the recess 76. On the other side of the fluid coupler body 70, as shown in FIGS. 5A and 5C, the gas delivery opening 80 is adjacent a locating recess 82. The locating recess 82 provides a tapered area for facilitating delivery of the testing gas to the gas sensor assembly 22. A purpose of the wider to narrower taper (FIG. 5A) of the locating recess 82 is to capture a top portion of the gas sensor in the fluid coupler body 70 as the latter is slid over the gas sensor. A tapered ramp portion 83 extends from the edge of the fluid coupler body and ends in a small generally flat semi-circular sensor engaging portion or area 84. A purpose of the ramp portion 83 is to allow the gas sensor to engage and capture the fluid coupler body 70 on the ramp rather than jamming against the edge of the fluid coupler body. When fully engaged or coupled, the gas sensor has traveled all the way up the ramp portion 83 and is firmly seated (FIG. 5C) against the sensor engaging portion 84 so that the gas sensor 22 is centered under the gas delivery opening 80. The resiliently deformable plastic fluid coupler body 70 is pressed away from the gas sensor, but owing to its resilient nature remains against the surface of the gas sensor due to the resilient nature of the fluid coupler body 70. Because of the slope of ramp portion 83 (FIG. 5C), a space or gap 100 exists above the gas sensor 22 to allow the testing gas to escape and activate the gas sensor. As a result, the gap 100 will remain generally repeatable for subsequent tests. This also ensures that the gas sensor is not sealed to the fluid coupler body 70 and that the test gas flows over the gas sensor to the edge of the fluid coupler body 70 for each test. In this manner, there is very little air to purge and the gas sensor can almost immediately react to a constant level of the testing gas. The gas delivery opening 80 and the tapered recess 82 are, in one embodiment, sized to be in overlying relationship and alignment with the gas sensor assembly. Other configurations and structures are envisioned for insuring the alignment and spacing of the gas delivery opening to a position proximate the gas sensor assembly as well for ensuring that the fluid coupler body does not jam against the gas sensor.

In the illustrated embodiment, the gas sealing member 78 is secured by an adhesive material 85 to the fluid coupler body 70. It will be appreciated that the recess 76 and gas opening 80 are arranged on the fluid coupler body 70 to be substantially aligned immediately adjacent or proximate the gas sensor assembly 22 (FIG. 7) when the fluid control body 70 is mated or otherwise coupled to the electronic control assembly and/or structure of the gas monitor apparatus. This advantageously insures testing gas being directly delivered to the gas sensor assembly instead of being applied to the exterior of the gas monitor. This promotes the purposes of efficient testing without wasting testing gas and reducing the amount of time for purging air.

The fluid coupler body 70 has an upstanding portion 86 provided with a curved stop segment or portion 88. The curved portion or stop segment 88 is sized and configured to engage a buzzer of the gas monitor apparatus 12 (see FIG. 7) and acts as a stop surface or segment for inhibiting rotational and lateral displacement of the fluid coupler body 70. In addition, a slot 90 extends along a portion of the fluid coupler body 70 that permits the fluid coupler body 70 to slide into engagement with a stop segment that engages one of the mounting posts 92 (FIG. 7) of the front cover assembly 32. The end of the slot 90 provides a stop segment that limits displacement and provides alignment of the gas delivery opening relative to the gas sensor. As such, the fluid coupler body 70 is prevented or stopped from sliding laterally in one direction (downward, as viewed in FIG. 7). In the illustrated embodiment, the fluid coupler body 70 is provided with a series of spaced apart stop projections 94 on one end of a leg portion thereof. The stop projections 94 extend exteriorly from the mated front cover and back plate assemblies to thereby stop at least longitudinal sliding movement of the fluid coupler body 70 in an opposite direction (i.e., rightward, as viewed in FIG. 1). Other equivalent structure can be provided so as to limit or stop displacement of the fluid coupler body 70. As noted, this further prevents unwanted movement of the fluid coupler body 70 during the CO testing process. Hence, the tendency for unwanted sliding movement that may be caused by the weight of the gas canister 18 and the regulator 20 tugging or pulling on the fluid coupler body 70 during testing is minimized or avoided. Accordingly, there is a more secure testing environment insuring proper delivery of testing gas.

The fluid coupler body 70 is, as noted, to be mounted to the gas monitor apparatus 12 after the front cover assembly 32 is moved as by the legs 69 pivoting or otherwise moving relative to the openings 55 in the back plate assembly to an open position. Attachment of the fluid coupler body 70 is easily and quickly achieved because the fluid coupler body is constructed in a manner that provides a relatively high degree of certainty that the gas delivery opening 80 is properly aligned immediately adjacent the gas sensor assembly 22. Such relatively precise alignment optimizes the CO testing process thereby minimizing false readings. In addition, since the gas delivery opening is aligned and immediately adjacent the gas sensor assembly, the latter is exposed directly to the testing gas in a manner that reduces the need to purge air surrounding the gas sensor assembly. Accordingly, the gas sensor assembly experiences, relatively quickly, gas at a concentration level used for the testing, whereby testing at the desired gas concentration level may commence. Moreover, the present disclosure envisions that the fluid coupler body 70 may slide into an opening or slot (not shown) formed in a side of the gas monitor housing instead of having to open the front and back assemblies.

### Electronic Control Assembly

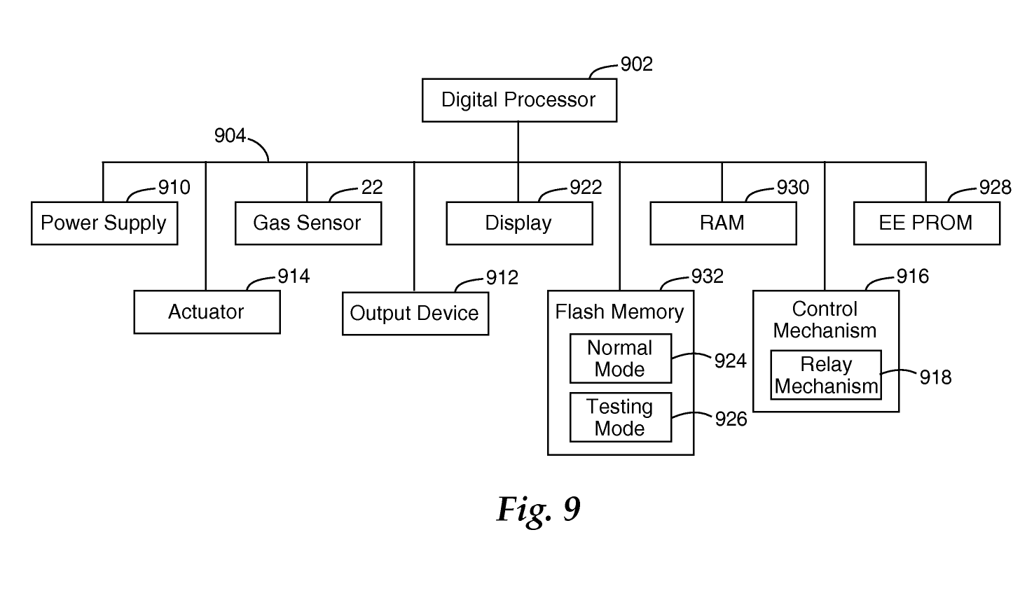

FIGS. 4, 7 and 9 illustrate aspects of an electronic control assembly 900. FIG. 9 is a simplified block diagram of an electronic control assembly 900 attached in spaced apart relationship to an interior surface of the front cover assembly 32. When the front cover assembly 32 is pivoted to its open condition, the fluid coupling apparatus 16 can then be easily and directly attached to the electronic control assembly 900 as illustrated in FIG. 7 to deliver the testing gas directly thereto.

In an exemplary embodiment, provision is made for a digital processor 902, such as, for example, a microcontroller, to be coupled to an information system bus 904. The information system bus 904 interconnects with the other components of the electronic control assembly 900. In an exemplary embodiment, the electronic control assembly 900 including the gas sensor assembly 22 may be mounted on a printed circuit board assembly 908. The gas sensor assembly 22 can be any suitable type. Typically, a semiconductor kind is utilized for monitoring CO gas in commercial units. More typically, the semiconductor gas sensor assembly 22 may be commercially available from Figaro USA Inc. of Glenview, Ill. Other suitable CO sensors are envisioned for use. As noted, the present disclosure is applicable for testing monitors for other gases as well. Hence, other types of gas sensors would be used.

The electronic control assembly 900 includes an output device 912, such as a buzzer unit 912 mounted on the printed circuit board assembly 908. The buzzer unit 912 operates to provide audible warning sounds to an operator/user in response to inappropriate levels of CO gas being detected by the gas sensor assembly 22. Other suitable output devices 912 that issue warnings in any desired manner are contemplated for use, for example, visual indicators (e.g., light-emitting diodes, etc.), third party alarm systems, display devices or the like.

An actuator switch 914 is mounted on the printed circuit board assembly 908. A distal end of the switch actuator rod 66 is spaced from a surface of the actuator switch 914. The actuator switch 914 is adapted to be contacted by the end of a switch actuator rod 66 and, as will be described, functions to initiate both the normal mode of operation and the CO testing mode process of this disclosure depending on the number of times the actuator switch 914 is actuated. Other suitable actuation schemes are contemplated. In the present embodiment, a single switch is used for effecting normal and testing modes. However, other switching arrangements may be utilized to implement such modes of operation.

A control mechanism 916 includes a relay mechanism 918 which operates under the control of the digital processor 902. The relay mechanism 918 is used to send a signal to an external alarm device on a monitoring panel (not shown). Under the control of the digital processor 902 and in response to sensed conditions by the gas sensor assembly 22, in a normal operating mode, the digital processor 902 sends signals to activate, for example, the buzzer unit 912 that predetermined levels CO gas concentrations considered potentially harmful are present. The digital processor 902 may also provide other signals, such as when a replaceable battery (not shown) is running low. A power supply 910 is provided for providing power for the electronic control assembly 900. The power supply 910 may be hardwired and/or be a replaceable battery (not shown) to be housed in the gas monitor apparatus 12. The power supply 910 may be coupled to the wiring harness 40. The digital processor 902 (e.g., microcontroller) may act to control operation of a display 922 (e.g., light-emitting diode 922) in a known manner through display signals. In this embodiment, the display is a single element, but may be implemented in with any suitable display or number of displays. The signals of the light-emitting diode 922 may be manifested by different colors that flicker and/or are constant and their states are selected to be representative of certain desired operating conditions. Other similar and well-known implementations for providing displays indicative of different states of the gas monitor apparatus are envisioned. The light-emitting diode 922 is adapted to be in registry with the display opening 68 (FIG. 2).

The digital processor 902 may be any suitable type. The digital processor 902 is attached to the printed circuit board assembly 908. The digital processor 902 is programmed to be responsive to monitored testing gas parameter readings obtained by the gas sensor assembly 22 performed over one or more time intervals for monitoring performance of the gas monitor apparatus 12. As noted, in this embodiment, the digital processor 902 is implemented as a microcontroller, such as is available from Microchip Technology Inc. of Chandler, Ariz., USA. The digital processor 902 may also be implemented in hardware, such as an Application Specific Integrate Circuit (ASIC) on a semiconductor chip. The digital processor 902 is preprogrammed with suitable applications to perform the normal mode operations mentioned above, but also the testing mode operation as described below.

The digital processor 902 sends and receives instructions and data to and from each of the system components coupled to the interconnect bus 904 to perform system operations based on the requirements of firmware applications that include a firmware application 924 for normal mode operation of the gas monitor apparatus and a testing mode firmware application 926. These firmware applications 924 and 926 may be stored in a permanent or non-volatile memory device, such as flash memory 932, or some other suitable non-volatile memory device(s) that would be appropriate for the data being handled. The program code of the firmware applications 924 and 926 are executed from the flash memory 932 under control of the digital processor 902. The random access memory (RAM) 930 is used to store the data during firmware execution. While the testing mode application 926 is implemented as firmware executable by the processing unit, it may be implemented as hardware (e.g. circuitry). The testing mode application operates the digital processor 902 to activate the display 922 for indicating pass/fail conditions. An electrically erasable programmable read only memory (EEPROM) 928 may also be used and contains other data, such as the predefined parameter values associated with the operating characteristics of the gas sensor assembly 22 as described below.

FIG. 8 illustrates a sensor response graph 800 of a series of individual curves 802ₐ₋ₙ (collectively 802) plotted from a series of previous sample tests generated by gas sensors of the type that fall within a group or class of sensors to which the present gas sensor assembly 22 is similar (e.g., semiconductor sensors) and which have been validated. In this embodiment, the predefined parameter values with which the response of the gas sensor assembly 22 is to be validated against are the values associated with a selected one of the gas sensor response curves 802, as will be explained. According to this disclosure, it was determined that the curve 802 with the lowest slope (e.g. 802ₙ) as viewed in the gas sensor response graph 800 is one that is considered to represent the slowest response time of an otherwise acceptable operating gas sensor that has been validated. The response curves generated after long gas exposure are considered to have the slowest response time. As such, the slowest acceptable response curve may be selected for purposes of comparing to the gas sensor assembly 22 for validation purposes. Alternatively, a sensor response graph may be generated based on previous validation responses of the actual gas sensor assembly 22 instead of being compared to a group of similar sensors.

In this alternative example, the response curve that is the lowest (lowest slope), as viewed in a response graph (FIG. 8) may be selected to yield a response curve that has the slowest response that would otherwise validate the response of the gas sensor assembly 22. It will be appreciated that the slowest or less responsive curve is used for defining one limit or boundary of acceptable gas monitor performance. As will be described below other response curves (e.g., the fastest or most responsive) may be used and which define another limit or boundary of acceptable gas monitor performance according to this disclosure.

The graphs generated are exemplary of many that may be used. It may further be appreciated that a sensor may not have the same response to a particular gas if some environmental conditions change. There are many uncontrolled variables that affect sensor responses. For example, variables like humidity, temperature, and a natural spread of readings in a group of monitors also affect a response curve. Thus, it will be appreciated that the curves presented herein can change based on such a wide number of variables. Nevertheless, according to the present disclosure, at least one of a series of generated curves can be selected and used for comparison purposes in the manner described below. In an illustrated embodiment, the curve selected may reflect the slowest acceptable response. As will be explained below, other sensor response curves to CO could be obtained, such as a typical first exposure to gas response (fastest or most responsive type of curve). Responses at different levels of testing gas concentration (e.g., 100 ppm, etc.) can also be utilized.

As noted, the curve 802ₙ is considered to represent a response that is close to the slowest response of a properly functioning gas sensor. This is considered satisfactory for validating the gas sensor assembly 22. The slope or rate-of-rise of the sensor response curve 802ₙ indicates a rate-of-rise of values or slope that will lead to an equilibrium response or equilibrating state of the gas sensor assembly within a predetermined time interval considered validating by, for example, a manufacturer. As noted, “equilibrium response” used in the specification and claims defines a response, such that gas reading values of the gas sensor assembly 22 of the gas monitor apparatus 12 being tested no longer increases. According to this embodiment, the curve 802ₙ has been used to define a predetermined rate-of-rise value used for comparison purposes for validation. As such, it will set one of the two bounds of acceptable gas monitor performance. The predetermined rate-of-rise value is obtained after a predetermined time has elapsed (e.g., one (1) minute) following the gas sensor value obtaining a reading or threshold value of 30 ppm (the threshold value is the validating rating of the gas sensor assembly 22 being tested). The point 804 on the response curve 802ₙ indicates a sensor reading after the predetermined time (i.e., 1 min.) has elapsed following the threshold value being reached. As an example, the value at point 804 is a reading of 170 ppm. The predetermined rate-of-rise value is computed by taking the value of 170 ppm and subtracting 30 ppm (validating or threshold value of the gas sensor). After such computation, the difference measures 140 ppm. Since the predetermined time interval is one (1) minute, the rate-of-rise is 140 ppm/minute. Other suitable time intervals can be utilized for determining the slope.

To provide a safety factor in order to be conservative, the value of 140 ppm/minute was multiplied by a safety factor of 50%. It should be-understood that the safety factor value of 50% is selected for this gas monitor, but that the safety factor value may be different for other devices and/or as more data becomes available. The approach taken in this embodiment is to establish bounds for an acceptable response of a gas sensor to pass the test. Acceptable safety factor values might be in a range of greater or lesser than 50% according to this disclosure. Safety factor values utilized for defining the bounds of the slowest response curve take into account known variables that affect response times of sensors. In this manner, the predetermined rate-of-rise value will not cause a failure reading when in fact none exists. It will be appreciated that a wide range of acceptable safety factor values might be utilized and these examples should not be considered limiting.

Referring back to FIG. 8, if the gas sensor assembly is later tested and has a rate-of-rise value at least reaching at least 70 ppm/minute, such will indicate that the gas sensor assembly has “passed” the test and is considered operable in the intended manner. Alternatively, if a test rate-of-rise value is less than 70 ppm/minute, then the gas sensor assembly will “fail” the test and be considered inoperable for the purposes intended. While, the exemplary value of 70 ppm/minute is selected other suitable values can be selected. For example, the rate-of-rise value can fall within a band or range determined to be accepted for residential and commercial use.

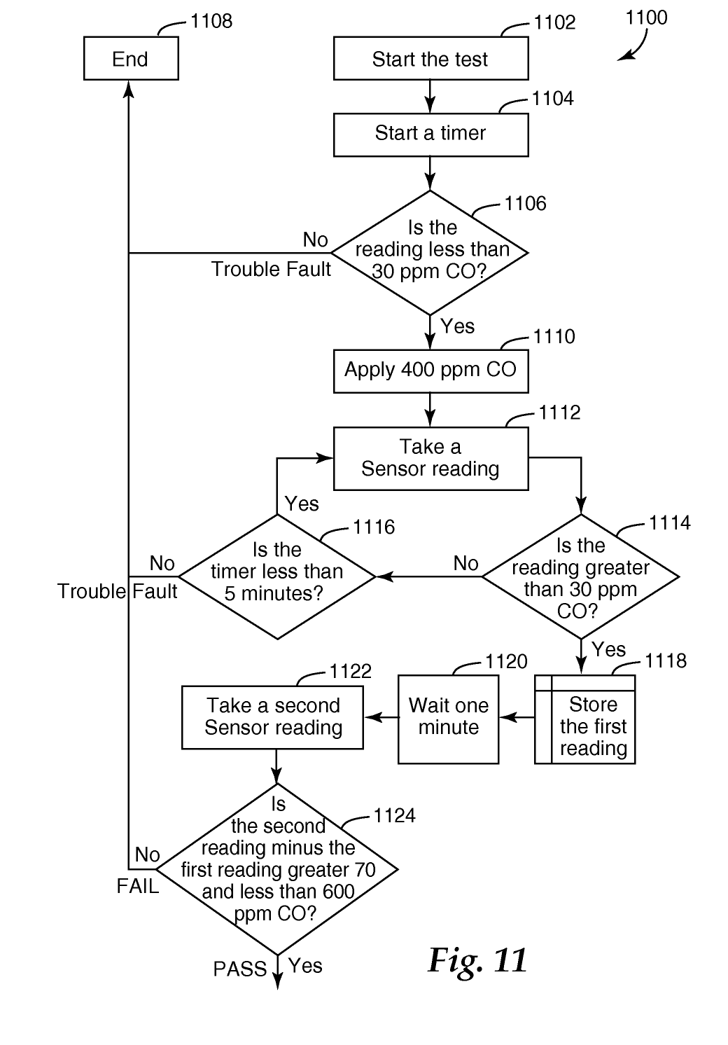

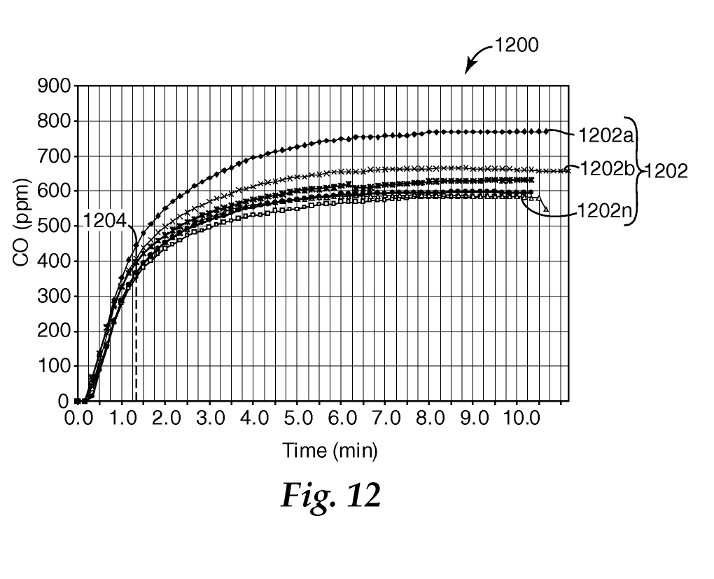

Other factors may cause the gas sensor assembly 22 to alarm prematurely. Sensors typically fail manufacturer or industry standards if they react too slowly, or too fast. For example, a gas sensor assembly may respond prematurely fast (outside the bounds of acceptable performance) if a resistor (not shown) of the electronic control assembly malfunctions. Therefore, the present disclosure contemplates having a second predetermined rate-of-rise value that can be compared against to see if the gas monitor apparatus properly functions. This will be explained below. In this regard, reference is made to FIGS. 11 and 12 for illustrating how a second predetermined rate-of-rise value is generated.

The monitoring application defines a gas testing process 1000 that will validate the gas sensor assembly 22 being validated. Essentially, the monitoring application, awaits initiation of the testing mode. This is achieved after the actuator switch is activated by a user. In this embodiment, the actuator switch 914 is rapidly and sequentially activated within several seconds by the user to commence the testing mode by the testing mode application 926. Such a signal differentiates its function relative to other functions that may be initiated by the switch.

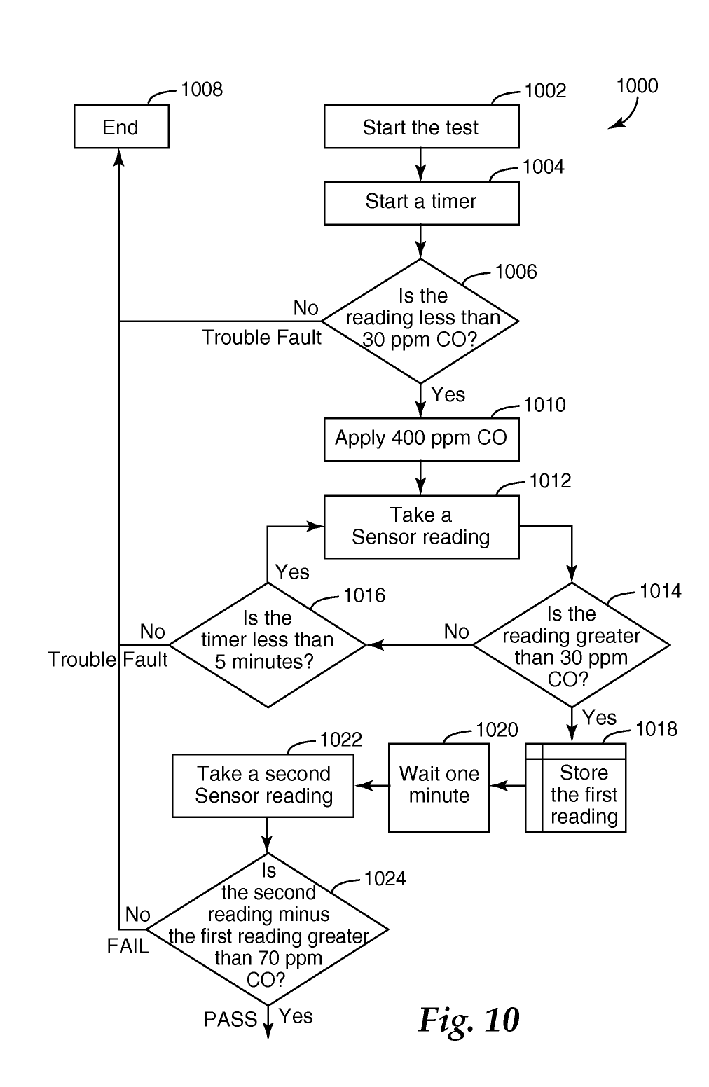

Reference is now made to FIG. 10 for illustrating one embodiment of a gas testing process 1000 implemented by using the gas monitor apparatus testing mode application 926 according to the present disclosure. In block 1002, the gas testing process 1000 commences. A test administrator or inspector will attach the fluid coupling apparatus 16, with the tubing 24 attached to the regulator 20, to the electronic control assembly 900 as described above wherein the gas delivery opening is aligned with the gas sensor assembly. As a result, the testing gas can be sensed by the gas sensor assembly 22 when actually applied as will be explained below. The testing gas utilized has a concentration selected to trigger the alarm. For example, the testing gas has a concentration of 400 ppm which not only exceeds the concentration response of the gas monitor apparatus 12 (e.g., 30 ppm) utilized but also insures a quicker testing process. Other concentrations of testing gas may be utilized to test the monitor. Generally, the lower the concentration of gas utilized for testing the longer the test.

According to this embodiment, it is desired that prior to running the testing process 1000, the air surrounding the gas monitor apparatus 12 should be clear of concentrations of carbon monoxide that exceed the minimum concentration response (e.g., 30 ppm) of the gas monitor apparatus 12. Towards this end, the testing process 1000 proceeds to start timer block 1004 whereby the gas sensor assembly 22 obtains a first reading. Following obtaining the first reading, the testing process 1000 proceeds to a decision block 1006, whereat a preliminary determination is made as to whether or not the air surrounding the gas monitor apparatus is clear of concentrations higher than the minimum concentration value (e.g., 30 ppm)of the gas monitor apparatus in order for the testing process 1000 to pass.

If the determination is negative (i.e., No) that the reading value does, at least reach the minimum concentration response of 30 ppm then such is indicative that the air surrounding the monitor is not as clear as desired. Hence, a trouble fault is recognized at a fault block 1008 which thereby ends the testing process. As such, the tester or user will try to clear the air surrounding the gas monitor. Alternatively, if the decision in the decision block 1006 is affirmative (i.e. Yes) then the testing process 1000 proceeds to the apply gas block 1010, whereat the tester or user opens the regulator 20 to allow carbon monoxide to travel to the fluid coupler body 70.

Following the application of the testing gas, the testing module obtains another reading which is taken by the gas sensor assembly 22 at the take sensor reading block 1012. At decision block 1014, a determination is made as to whether or not this previous reading at least reaches a threshold value that is related to the response of the gas sensor assembly. In the illustrated embodiment, 30 ppm is considered the threshold value which is the minimum concentration response of the gas monitor apparatus 12. If the determination in the decision blocks 1014 are negative (i.e., No), the testing process 1000, and then proceeds to the decision block 1016 whereat a decision is made if the timer has been running for less than five (5) minutes. In particular, at the decision block 1016, if a determination is made that the timer has been running for less than five (5) minutes then the testing process 1000 loops back to take a subsequent sensor reading block 1012. Other reasonable times are contemplated besides five (5) minutes. The testing process 1000 will continue this loop until either the decision in the block is indicative of a reading that the gas sensor assembly has read a value that at least reaches 30 ppm or the timer has exceeded five (5) minutes of running time and the read value has not at least reached 30 ppm. In the latter case, the testing process 1000 proceeds to the fault block 1008 to indicate that the gas reading is indicative of the fault condition whereby the testing process 1000 terminates.

If the decision of the decision block 1014 is affirmative (i.e., Yes) then the testing process 1000 stores this first reading in the reading store block 1018 in the RAM memory. Thereafter, the testing process 1000 introduces a time delay of a predetermined time by a time delay block 1020 for enabling the taking of a second reading by the gas sensor assembly in the second reading block 1022. In the illustrated embodiment the time delay introduced by the time delay block 1020 is one minute. Of course, other time delays may be utilized depending on the nature of the gas being tested.

Following the second reading, after the predetermined time interval, the testing process 1000 then proceeds to the decision block 1024. In the decision block 1024, testing module application 926 is utilized to predict if the minimum concentration response of the gas sensor assembly after 1 minute at least reaches a predetermined rate-of rise parameter value (e.g. 70 ppm/minute). Hence, the testing module application 926 determines if the monitor is operative or not within a short period of time without having to the test for a typical testing period.

If the determination is affirmative (YES), then a passing condition (i.e., “passes”) of the gas monitor apparatus 12 is achieved by the testing process 1000. Alternatively, if the testing module application 926 determines that the gas monitor apparatus 12 does not at least reach the 70 ppm/minute then the testing process 1000 proceeds to the fault block 1008, whereby the testing process ends. This is indicative of the gas monitor apparatus 12 not passing the test.

Reference is made to FIGS. 11 & 12, for describing an alternate embodiment of the present disclosure. Initial reference is made to FIG. 12 which illustrates a sensor response graph 1200 of a series of individual curves 1202ₐ₋ₙ (collectively 1202) plotted from a series of previous sample tests generated by gas sensors of the type that fall within a group or class of sensors to which the present gas sensor assembly 22 is similar (e.g., semiconductor sensors) and which have been validated. In this embodiment, the predefined parameter values with which the response of the gas sensor assembly 22 is to be validated against are the values associated with a selected one of the gas sensor response curves 1202, as will be explained. According to this disclosure, it was determined that the curve 1202 with the highest slope (e.g. 1202ₐ), as viewed in the gas sensor response graph 1200, is one that is considered to represent the fastest response time of an otherwise acceptable operating gas sensor that has been validated. In taking into account the different response characteristics of gas monitors, the present embodiment selected typical responses of a gas sensor that have not been exposed to CO for a considerable period of time. Unlike the response curves noted above that were generated after long gas exposure, these are generated following first exposure of a sensor to the gas. As used in the present application “first exposure” is considered to be the first exposure of the sensor to gas after a prolonged time that the sensor has not sensed gas. The prolonged time period may be, for example, as short as four (4) weeks or longer. As such, the fastest acceptable response curve may be selected from one of these response curves for purposes of comparing it to the response of the gas sensor assembly 22 for validation purposes of the upper limit to an acceptable range of performance. Alternatively, a sensor response graph may be generated based on previous validation responses of the actual gas sensor assembly 22 instead of being compared to a group of similar sensors.

As noted, the curve 1202ₐ is considered to represent a response that is close to the fastest response of a properly functioning gas sensor. This is considered satisfactory for validating the gas sensor assembly 22. According to this embodiment, the curve 1202ₐ has been used to define a predetermined rate-of-rise value used for comparison purposes for validation. As such, it will set one of the two bounds of acceptable gas monitor performance. The predetermined rate-of-rise value is obtained after a predetermined time has elapsed (e.g., one (1) minute) following the gas sensor value obtaining a reading or threshold value of 30 ppm (the threshold value is the validating rating of the gas sensor assembly 22 being tested). The point 1204 on the response curve 1202ₐ indicates a sensor reading after the predetermined time (i.e., 1 min.) has elapsed following the threshold value being reached. As an example, the value at point 1204 is a reading of about 427 ppm. This is the value of a reading 60 seconds later than a 30 ppm reading (validating or threshold value of the gas sensor assembly). The predetermined rate-of-rise value is computed by taking the value of 427 ppm and subtracting 30 ppm (validating or threshold value of the gas sensor assembly 22). After such computation, the difference measures 397 ppm. Since the predetermined time interval is one (1) minute, the rate-of-rise is 397 ppm/minute. Other suitable time intervals can be utilized for determining the slope.

If we use a 150% safety factor, the maximum rate of rise is (427−30)*1.5=596 ppm/min. This has been approximated to 600 ppm/minute. Acceptable safety factor values might be in a range of greater or lesser than 150% according to this disclosure. Safety factor values utilized for defining the bounds of the fastest response curve take into account known variables that affect response times of sensors. In this manner, the predetermined rate-of-rise value will not cause a failure reading when in fact none exist. It will be appreciated that a wide range of acceptable safety factor values might be utilized and these examples should not be considered limiting.

FIG. 11 represents another testing process 1100 according to this disclosure. This embodiment presents an embodiment wherein first and second predetermined rate-of-rise values are utilized to define bounds or a range of acceptable validating performances of the gas monitor apparatus 12. The testing process 1100 is similar to the testing process 1000 described above. In particular, the blocks 1102-1122 perform substantially the same processes as those described above in corresponding blocks 1002-1022. Hence, a discussion of the functions of the blocks 1102-1122 is not presented herein. A difference between the testing process 1100 and the testing process 1000 is that in block 1124, first and second predetermined rates-of-rises are used to define lower and upper bounds or range of acceptable validating performance. Thus, the testing module application 924 includes the functions of the block 1124 which will be described below in the context of FIG. 12. In the decision block 1124, testing module application 926 is utilized to predict if the minimum concentration response of the gas sensor assembly after 1 minute at least reaches a first predetermined rate-of rise parameter value (e.g. 70 ppm/minute) for one limit or bound (e.g., slowest response considered acceptable) and if it does not exceed a second predetermined rate-of-rise value of 600 ppm/minute for another limit or bound (e.g., fastest response considered acceptable) of an acceptable range of performance. Hence, the testing module application 926 determines if the monitor is operative or not, within a short period of time, without having to test for typical testing period. For instance, with 400 ppm, testing may be accomplished either in about or less than 1½ minutes. If an equilibrium test were conducted, as noted above, on a gas sensor being used in the present illustrated embodiment, the sensor could be validated in about 4.5 to about 5 minutes (or about at least 300% more time). Hence, the testing reduces significantly the testing time.

As such if the determination is affirmative (YES) in the block 1124 then the gas monitor apparatus 12 “passes” the testing process 1100. Accordingly, for a passing condition to exist, the rate-of-rise value during the test must at least reach 70 ppm/minute and must not exceed 600 ppm/minute. Alternatively, if the testing module application 926 determines that the gas monitor apparatus 12 exceeds the 600 ppm/minute then the testing process 1100 proceeds to the fault block 1108, whereby the testing process 1100 ends. This is indicative of the gas monitor apparatus 12 not passing or failing the test because its response is either too fast or slow based on a comparison with the bounds of acceptable gas monitor performance.

FIGS. 13-17 are illustrative of one exemplary embodiment of a portable hand-carried testing tool or portable gas testing system 1300 of this disclosure. The gas testing system 1300 is adapted to be coupled to one or more gas monitors 1302; only the interior of a front cover assembly 1304 thereof is illustrated in FIG. 13. It will be understood, however, that the gas monitors that the gas testing system 1300 are used in combination with are similar to the one described above. Alterations of the above gas monitors have been made so as to carry out the process of this embodiment. Such alterations will be described below. Since the gas sensor evaluation is performed with the portable gas testing tool or system 1300 rather than in a fixed environment (i.e., within a gas monitor), a highly mobile and versatile gas testing process may be implemented. Accordingly, the portable testing tool may be carried from one gas monitor to another. The gas testing system 1300 performs data processing of gas sensor data gathered from the gas monitor using, in essence, the testing mode application or testing module application discussed above. However, alterations of the prior testing module have been made and are set forth hereinafter in order to describe its operation in a portable environment.

A separate fluid coupler 1306 is provided that is similar to the one described above for delivering testing gas that may be used in performing a gas testing process of this embodiment. As such, a detailed description of its structure and functions are described, supra. While the fluid coupler 1306 is illustrated as being a separate element, this disclosure envisions that the fluid coupler 1306 and the gas testing system 1300 may be integrated as a single unit. Alternatively, the testing gas may be applied by other devices than the fluid coupler and yet the portable features of the gas testing system 1300 are not affected.

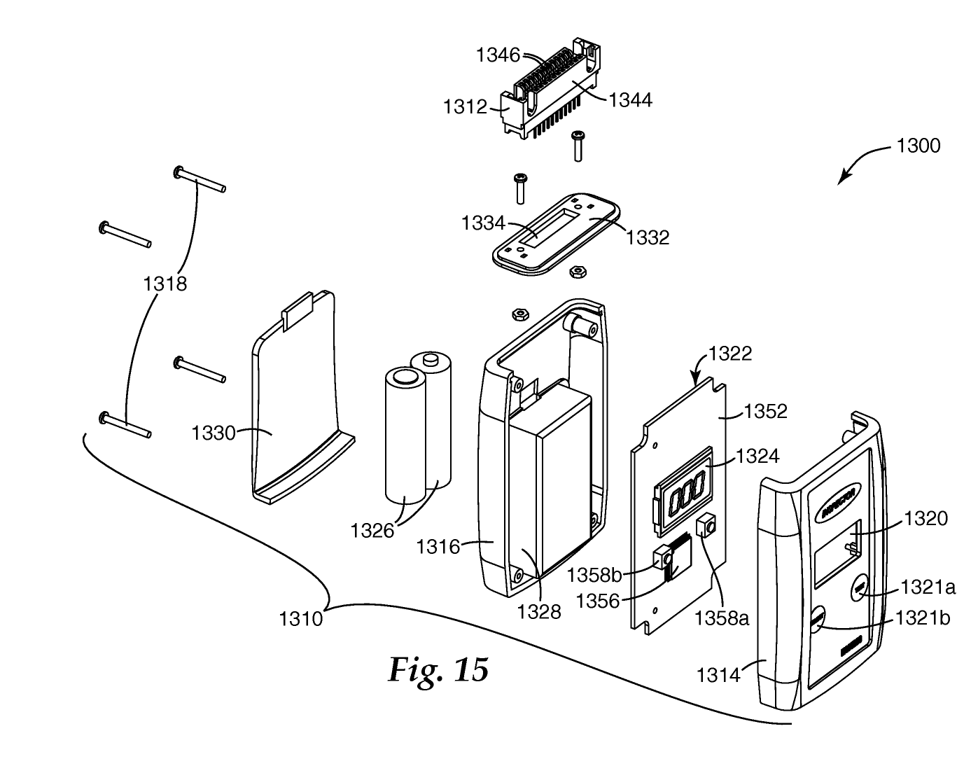

Continued reference is made to FIGS. 13-17 for illustrating one exemplary embodiment of the portable gas testing system 1300. The gas testing system 1300 includes a portable housing assembly 1310 having a printed circuit type card edge connector assembly 1312 protruding from one end thereof. Referring to FIG. 15, the housing assembly 1310 includes generally rectangular front and back cover assemblies 1314 and 1316 that are mated together (see FIGS. 13 & 14). The front and back cover assemblies 1314 and 1316 may be secured together by threaded members 1318 (FIG. 15) that fit within appropriate passages and threaded bosses or the like of the housing assembly. A display opening 1320 is provided in the front cover assembly 1314. A pair of switch buttons 1321a and 1321b is also provided. A wide variety of housing assembly constructions and configurations are embraced by the spirit and scope of this disclosure. While this embodiment describes the front in the orientation as illustrated, it will be appreciated that the front cover assembly may be oriented in any suitable side including facing outwardly with respect to the front cover.

An electronic control assembly 1322 (FIGS. 15 & 16) is included within the housing assembly 1310 and is operable for implementing the gas testing process of this disclosure as will be described. Included in the electronic control assembly 1322 is a display device 1324 that may be any suitable type, such as a liquid crystal display (LCD) device 1324 that provides for alphanumeric readouts. The LCD display device 1324 is in registry with the display opening 1320. Although a LCD display device is illustrated, other suitable visual displays or other information output devices may be used. In this embodiment, the LCD display device 1324 is a single unit, but may be comprised of several LCD units.

A battery power supply 1326 for the electronic control assembly may include a pair of alkaline or rechargeable batteries 1326 (FIG. 15) housed within a battery compartment 1328 and is used for providing the power necessary for operating the gas testing system 1300. A removable battery door 1330 is connected to the back cover assembly 1316 for allowing insertion and removal of the batteries 1326. A top panel 1332 is secured to the mated front and back housing assemblies 1314 and 1316 and acts to secure the card edge connector assembly 1312 thereto. An opening 1334 is formed to hold the card edge connector which has connector pins that cooperated with mating structure (not shown) on the printed circuit board. The opening 1334 is formed in the top panel 1332 to hold the card edge connector assembly 1312 so as to allow its other end for mating cooperation with the electronic control assembly 1336 (FIGS. 13 & 14) of the gas monitor 1302.

The electronic control assembly 1336 of the gas monitor 1302 is connected to the front cover 1304 of the gas monitor. The electronic control assembly 1336 of the gas monitor 1302 essentially functions as the electronic control assembly of the gas monitors of the previous embodiments. However, as will be pointed out some changes have been made since the testing gas mode is carried out by a portable gas testing system and not the fixed monitor itself. Thus, for instance, there is no need for the above described testing mode application to be stored in the flash memory in the gas monitor's electronic control assembly 1336. In addition, the electronic control assembly 1336 may be configured to provide real time data readings of the gas sensor assembly 1338 as well as unique identifying data of the gas monitor. The unique identifying data may identify a particular gas monitor, such as by a serial number. The serial number data provides specific information as to a particular gas monitor in a network that is being tested. Other kinds of unique identifiers may be provided. Gas sensor readings may be provided as digital or analog signals. These data signals are carried by the information bus (not shown) to a plurality of spaced apart signal contacts 1340 (FIG. 13) positioned on the printed circuit board 1342. The gas sensor readings may be representative of the CO concentration levels being sensed. The signal contacts 1340 are located adjacent an edge of the printed circuit board 1342 so as to be physically coupled to card edge connector assembly 1312. In this manner, a one-way mode of communication is established for transferring data from the gas monitor to the gas testing system. While a one-way mode is described, a bidirectional mode may be implemented as will be described below in another embodiment.

Referring back to the printed circuit card edge connector assembly 1312, it may be any suitable type that is configured for physically coupling to the plurality of signal contacts 1340. Typically, the printed circuit card edge connector assembly 1312 may include a connector housing 1344 (FIGS. 13 & 14) defining a cavity that houses a plurality of connector contacts 1346. The connector housing 1344 is adapted to receive the edge of the printed circuit board 1342 in order to physically couple the connector contacts 1346 to the signal contacts 1340. A wide variety of suitable edge connector assemblies are envisioned for use. One typical type is commercially available from AMP Corp. of Harrisburg, Pa.

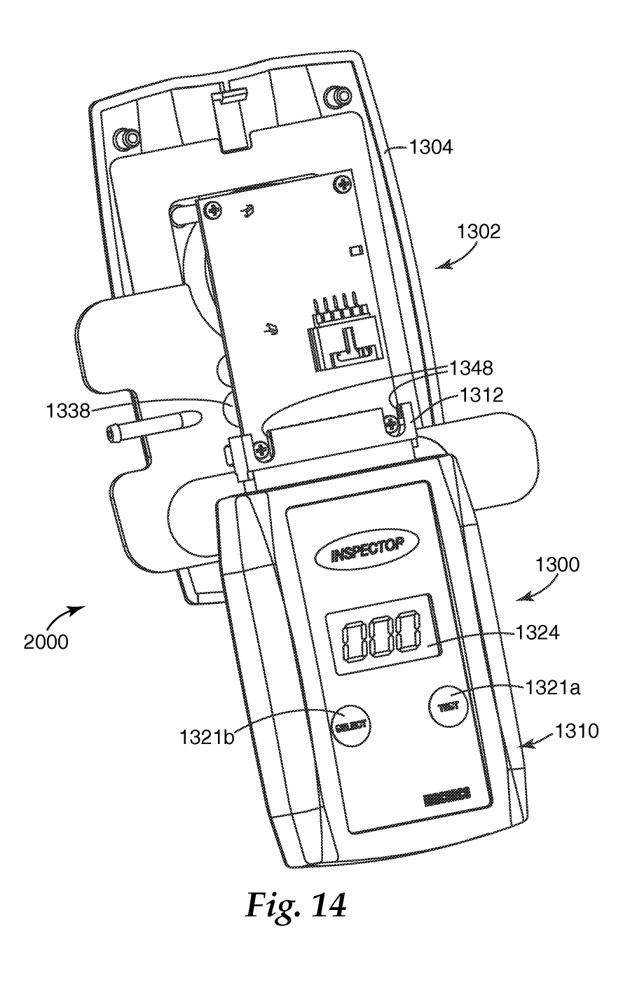

A pair of mating recesses 1348 is formed in the connector housing 1344 (FIG. 14). The mating recesses 1348 are sized and shaped to accommodate the mounting posts 1350 supporting the electronic control assembly 1336. In this manner, the mating recesses 1348 facilitate proper alignment of the signal contacts 1340 with respect to the data output signal contacts 1340. The card edge connector aligns itself to the printed circuit board and the connector contacts 1346 directly physically engage with the data output signal contacts 1340.

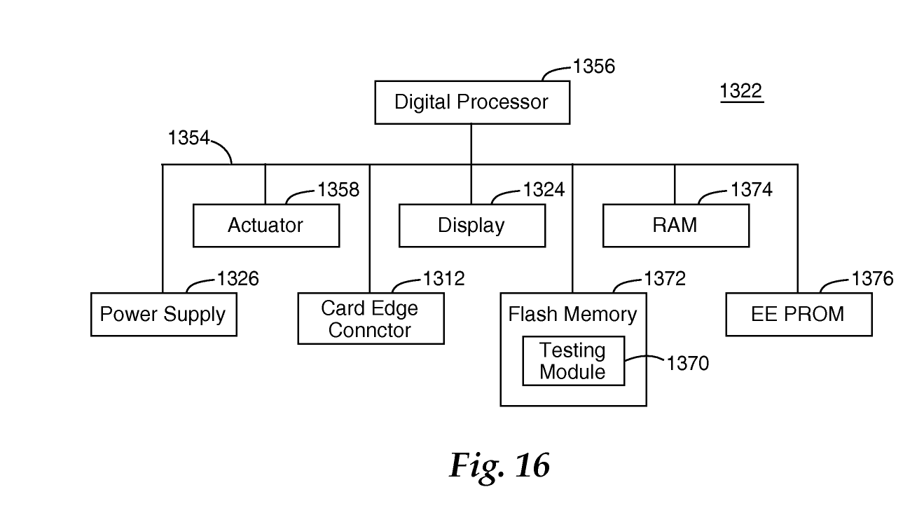

Referring to FIG. 16, a simplified block diagram of the electronic control assembly 1322 is illustrated that includes a printed circuit board 1352 (FIG. 15) of the portable gas testing system 1300. Included is an information system bus 1354 that interconnects with the components of the electronic control assembly 1322. In an exemplary embodiment of the electronic control assembly 1322, provision is made for a digital processor 1356, such as, for example, a microcontroller 1356 that is coupled to the information system bus 1354 and to the printed circuit board 1352. The display device 1324, the power supply 1326, and the printed circuit card edge connector assembly 1312 of the electronic control assembly 1322 are electrically connected to the information system bus 1354 as well. Also, connected to the information system bus 1354 is a test actuator 1358. The test actuator 1358 includes a test switch 1358a and a select switch 1358b (see, FIG. 15). The test and select switches 1358a and 1358b; respectively cooperate with the test and select buttons 1321a and 1321b; respectively, that protrude through corresponding openings formed in the front cover assembly 1314 (FIG. 15) whereby the former and the latter may cooperate together. A tester or user may manually activate the switches 1358a and 1358b in a manner to be described. While the exemplary embodiment describes use of a pair of switches for affecting the testing mode, other switching systems and arrangements may be utilized.

The digital processor 1356 may be any suitable programmable electronic device type. The digital processor 1356 is attached to the printed circuit board 1352. The digital processor 1356 is programmed to be responsive to monitored testing gas parameter readings transmitted thereto from the gas sensor assembly 1338. The readings may be obtained over one or more time intervals, for example, the data is provided at the rate of one per second. In this embodiment, the digital processor 1356 is implemented as a microcontroller, such as is available from Microchip Technology Inc. of Chandler, Ariz., USA. The digital processor 1356 may also be implemented in hardware, such as an Application Specific Integrate Circuit (ASIC) on a semiconductor chip. The digital processor 1356 is preprogrammed with suitable applications to perform the testing mode operations described below.

The digital processor 1356 may also provide other signals, such as when a replaceable battery 1326 is running low. The digital processor 1356 may act to control operation of the LCD display device 1324 in a known manner through display signals.

The digital processor 1356 may send and receive instructions and data to and from each of the system components coupled to the information systems bus 1354. The digital processor 1356 performs system operations based on the requirements of firmware applications including a testing module application 1370. The testing module application 1370 may be stored in a permanent or non-volatile memory device, such as flash memory 1372. Other suitable non-volatile memory device(s) may be used. The program code of the testing module application 1370 is executed from the flash memory 1372 under control of the digital processor 1356. A random access memory (RAM) 1374 stores the data during execution of the firmware applications. While the testing mode or testing module application 1370 is implemented as firmware executable by the digital processor 1356. It may be implemented as hardware (e.g. circuitry), or other programmable electronic devices, such as a computer system.

The testing module application 1370 operates the digital processor 1356 to activate the display device 1324 for providing different kinds of information useful for accomplishing the gas testing process. For example, information pertaining to a monitor's serial number, physical address, or providing a listing of monitors may be provided. Other information that may be provided includes peak CO level and elapsed time since the peak CO level. The latter may be useful in finding a detector that has gone into alarm. Accordingly, someone may want to test the detector that has gone into alarm to ensure that it is working correctly.

An electrically erasable programmable read only memory (EEPROM) 1376 may also be used that contains data, such as different test limits for different concentrations of gas or different test limits for different gases in the EEPROM. Also, a data log of the results could be stored in the EEPROM. This includes serial number data. These operating characteristics, as noted, above are used to validate operation of the gas sensors according to the testing module application. The EEPROM 1376 may also contain other data, such as data relating to unique gas monitor identifiers. An example of such an identifier is the serial number of each of the monitors. Each serial number identifies a corresponding one of the gas monitors for authentication purposes in the gas testing process. In addition, the data may include the physical addresses of each of the monitors or other suitable identifying information. As noted, the testing module application 1370 is configured to allow the tester or user to select a particular one of the gas monitors that may be listed in the display device 1324.

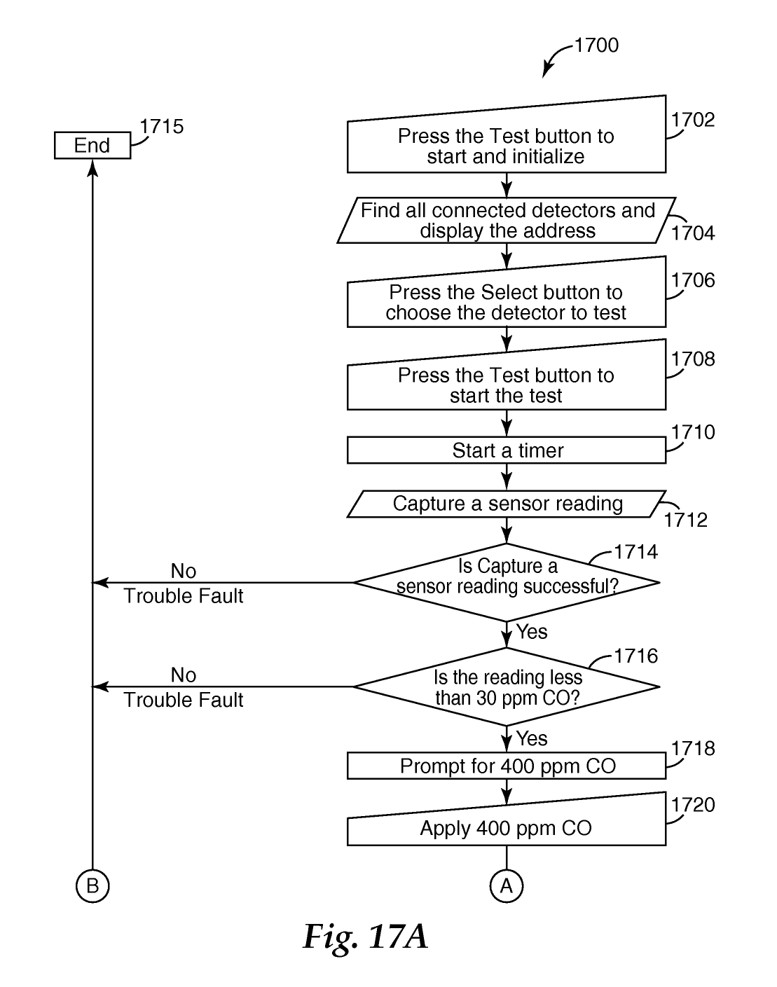

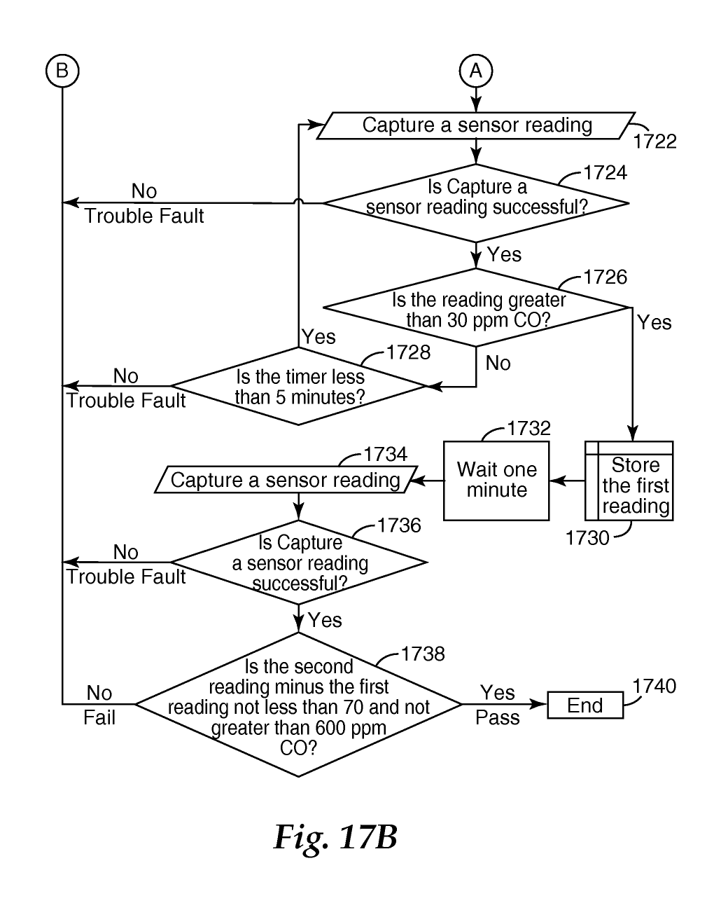

Reference is now made to FIGS. 17A & 17B for illustrating one embodiment of a gas testing process 1700 implemented by using the testing module application 1370. In a Press The Test Button To Start and Initialize block 1702, a tester or user may commence the gas testing process 1700 by pressing the test button 1321a, thereby actuating the test actuation switch 1358a. This action starts and initializes operation of the testing module application 1370 of the portable gas testing system 1300.

Thereafter, the gas testing process 1700 advances to the Find All Connected Detectors and Display The Address block 1704, whereat the gas testing process 1700 finds all gas monitors connected to the gas testing system 1300. As used in this application the term “connected” in this context means that a gas monitor is physically coupled to the gas testing system 1300. Alternatively, the term “connected” means that the gas monitors in a network are communicating, or the term “connected” means that a gas monitor(s) is wirelessly coupled to the gas testing system 1300. In a portable system that relies upon physical coupling, the gas monitor that is physically coupled is identified on the LCD display device 1324. Alternatively in a wireless system, the portable gas testing system 1300 may communicate with several gas monitors within its range of wireless communication. Hence, the digital processor 1356 may display in the LCD display device 1324 all those gas monitors found to be in proximity and communicating with the gas testing system 1300. The gas monitors so displayed may be displayed in an ordered manner. In this approach, the address of the first listed gas monitor may be displayed.

Several different approaches of displaying the information are contemplated. For example, such displayed information may include the physical address of each monitor. Accordingly, the tester or user may advance to those identified gas monitors in proximity to it for continued testing. In a network, the present disclosure envisions the testing tool or testing system facilitating selection of one of the gas monitors under the control of the testing module application 1370. To facilitate selection, a user or tester presses the test button to display the serial numbers of successive CO monitors. Once the correct serial number is displayed, the select button is pressed to test the chosen CO monitor.

In this regard, In Press The Select Button To Choose The Detector block 1706, the tester or user, activates as by pressing the select switch button 1321b to activate the select switch 1358b to thereby select which of the displayed gas monitors is to be tested further. Once selected, the tester or user then activates as by pressing the test button 1321a in the Press The Test Button To Start The Test block 1708 to commence testing according to the testing module application 1370. In the Start A Timer block 1710, a time interval under the control of the digital processor 1356, is started for carrying out the timing of the operations described hereinafter.

The gas testing process 1700 then advances to the Capture A Sensor Reading block 1712, whereat a gas sensor reading of a gas sensor assembly is captured by the gas testing system 1300. Of course, the noted gas sensor reading is transmitted to the gas testing system 1300 at the noted 1 (one) second intervals through the physical coupling noted above. In Is Capture A Sensor Reading Successful? decision block 1714, a decision is made as to whether or not a captured sensor reading is successful. By the term “successful” as used in the present application, it is meant that a determination is made as whether or not a gas sensor reading has been taken, regardless of the reading's value. Thus, the block 1714 does not evaluate any value associated with a sensor reading, but rather whether a gas sensor reading has in fact been taken or not. The relevance of the successful reading is to indicate that the selected gas monitor is operational and may be further tested. If the gas testing system 1300 does not capture a gas sensor reading, then the decision block 1714 indicates a trouble fault condition has arisen. As such, the gas testing process 1700 advances to an End of process block 1715. Alternatively, if the determination is affirmative (i.e., YES) in the decision block 1714 that a capture has been successful, then the gas testing process 1700 may continue as follows.

The gas testing process 1700 then advances to Is The Reading Less Than 30 ppm CO? decision block 1716. In this regard, the decision block 1716 makes a determination as to whether the gas sensor 1338 sensed gas having a concentration value of less than 30 ppm or not (the nominal operating level of the gas monitor). The gas testing process 1700 thereafter functions, as described above in regard to the block 1006 in FIG. 10, supra. Essentially, if the testing module application 1370 determines that the sensed gas concentration level is 30 ppm or higher at the gas monitor being tested, the testing module application 1370 issues a trouble fault signal. The trouble fault signal advances to the End process block 1715, thereby signifying the gas testing process 1700 should not advance since unclear air is present at the gas monitor. As noted, unclear air would not render a valid result. Alternatively, if the captured reading is less than 30 ppm, then the testing module application 1370 causes the display device 1324 to issue a suitable prompt at the Prompt For 400 ppm CO block 1718. The prompt advises the tester or user to apply the testing gas for continuing the testing. As in the previous embodiments, a testing gas of about 400 ppm is applied for purposes of continuing the gas testing process 1700. Also, as previously indicated other testing gas concentrations may be applied. The prompt may be implemented in a variety of suitable approaches besides as described above. In an Apply 400 ppm CO block 1720, the user or tester may apply the testing gas at the concentration level of 400 ppm to the gas sensor through the fluid gas coupler 1306 as described in the previous embodiments.

The gas testing process 1700 then advances to a Capture A Sensor Reading block 1722 (FIG. 17B). At the Capture a Sensor Reading block 1722, the testing module application 1370 is operable to capture another gas sensor reading. As earlier noted, the testing module application 1370 is operable at periodic time intervals to take such a reading. The time interval may vary, but in this embodiment, as noted the time interval is one second.

The gas testing process 1700 then advances to a Is Capture A Sensor Reading Successful? decision block 1724. In the decision block 1724, the testing module application 1370 is operable to determine whether or not the captured gas sensor reading was successful. The testing module application 1370 is not concerned with whether the captured reading has any particular value, but merely whether a value is present or not. If a reading was not captured (i.e., No), then the gas testing process 1700 indicates that a trouble fault condition exists (i.e., unsuccessful) and the gas testing process then advances to the End of process block 1715. Alternatively, if a captured reading occurs (i.e., successful) then the gas testing process 1700 advances to Is The Reading Greater Than 30 ppm CO? decision block 1726.

In the decision block 1726, the gas testing process 1700 determines whether the captured reading from the decision block 1724 is greater than 30 ppm CO. If the decision is negative (i.e., No) that the concentration level representative of the reading is not greater than 30 ppm, then the gas testing process 1700 advances to an Is The Timer Less Than 5 (five) Minutes? decision block 1728. The decision block 1728 decides if the captured reading occurred in less than five (5) minutes from the commencement of the timing as noted above. The five (5) minutes is selected since if the gas testing process takes five minutes or more there is likelihood that the gas testing process may not yield a valid result. For instance, the 5 minute time period is to prevent the test from going on indefinitely if there is no gas left in the test bottle, if for some other reason gas does not reach the sensor or if the sensor does not respond to the test gas. If the timer has run for five minutes or more then the gas testing process 1700 indicates a trouble fault. Hence, the gas testing process 1700 advances to the End of process block 1715. Alternatively, if less than five minutes has elapsed since commencement of the time period, a valid test is still possible. Accordingly, the testing module application 1370 loops back to the Capture A Sensor Reading block 1722, whereat another gas sensor reading is attempted to be captured. The gas testing process 1700 then returns to the Is Capture A Sensor Reading Successful? decision block 1724. In the decision block 1724, a decision is made as to whether the last gas sensor reading was actually captured or not. If a new sensor reading was not captured, then a trouble fault condition arises and the gas testing process 1700 then proceeds to the End of process block 1715. On the other hand, if a reading was captured, the gas testing process 1700 returns to the decision block 1726, whereat a decision is again made as to whether or not the reading is greater than 30 ppm CO. Thus, the gas testing process 1700 performed at the blocks 1724 and 1726 are repeated until either a trouble fault decision is made or the decision block 1726 determines in the affirmative that the gas sensor reading is greater than 30 ppm CO.

If the decision in the Is The Reading Greater Than 30 ppm CO? decision block 1726 is affirmative (i.e., YES) that the gas concentration value is greater than 30 ppm, then the gas testing process 1700 advances to a Store The First Reading block 1730, wherein the first reading from the block 1726 is stored in the RAM. Thereafter, the gas testing process 1700 advances to the Wait One Minute block 1732, and it waits for the next or second gas reading value. The waiting time period between the successful capture of a first reading and taking of a second reading is about 1 (one) minute. This is similar to the time interval noted above in regard to the other embodiments. Clearly, a different time interval may be used. However, for the sake of consistency one (1) minute is utilized. As noted earlier, the one minute time interval is selected to advance a quick and effective test. Following the one minute waiting period imposed by the Wait One Minute block 1732, the gas testing process 1700 advances to capture a second reading at the Capture A Sensor Reading block 1734. As noted previously, the testing module application 1370 is operated to capture the sensor reading. The second reading is a real-time gas concentration level of CO at the gas monitor following application of the 400 ppm CO.

After, the second reading is taken, the gas testing process 1700 advances to an Is Capture A Sensor Reading Successful? decision block 1736. A decision is made in the decision block 1736 as to whether or not a reading was obtained. If no such second reading is obtained, then the gas testing process 1700 indicates a trouble fault condition. Accordingly, the gas testing process 1700 advances to the End process block 1715. Alternatively, of course, if the second reading has been taken regardless of value, the gas testing process 1700 advances to the Is The Second Reading Minus The First Reading Not Less Than 70 ppm And Not Greater Than 600 ppm CO? decision block 1738.

The gas testing process 1700 carried out in the decision block 1738, determines is the second captured reading or captured value minus the first captured reading or captured value equal to or greater than 70 ppm or equal to or less than 600 ppm. If the decision is affirmative (i.e., Yes), then the gas testing process 1700 proceeds to End testing routine block 1740. Accordingly, the gas sensor assembly 1338 of the gas monitor being tested is considered validated or to have passed the testing process. Such information may be communicated to the LCD display device 1324 under the control of the digital processor. Alternatively, if the result of subtracting the first reading from the second reading falls outside the bounds of acceptable performance, then the gas testing process 1700 indicates a ‘FAIL’ condition, whereby the gas testing process advances to the End of process block 1715.

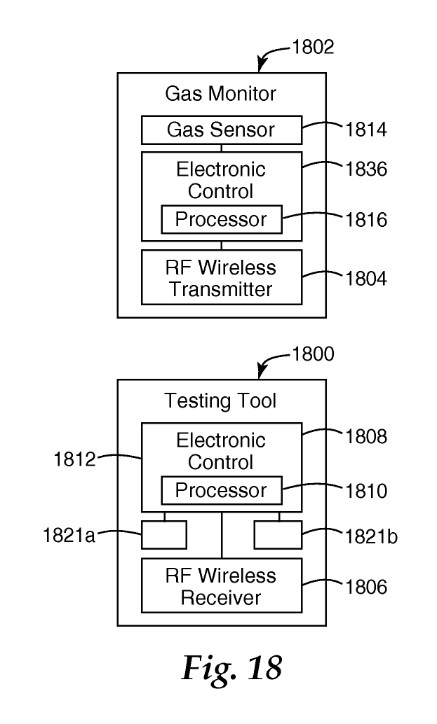

Reference is now made to FIG. 18 for illustrating an exemplary embodiment of a wireless portable testing tool 1800. The essential differences between this embodiment and the previous embodiment are that the relevant data and instructions are not transmitted directly by hard wire, but rather in a wireless mode. Accordingly, the testing tool 1800 is operable for wireless operation with a gas monitor 1802. The gas monitor 1802 is constructed to operate in much the same manner as the previous embodiment, with the main difference being that data and instructions are transmitted wirelessly rather than by a hardwire connection. As such, the gas monitor 1802 includes a wireless transmitter device 1804, such as radio frequency (RF) transmitter 1804. While radio frequency is described in one exemplary embodiment as the mode of wireless communication, other suitable modes of wireless communication are envisioned. For example, other envisioned forms of wireless communication include but are not limited to the following modes: infrared, microwave, acoustic, etc. Of course, according to this disclosure, it will be understood that the mode receiving the wireless data and instructions is compatible to the mode of transmission.

The portable testing tool 1800 includes a housing assembly 1808 that houses a wireless data receiver device 1806 that communicates with the wireless RF transmitter device 1804 in the gas monitor 1802. The RF receiver 1806 transfers the received signals through a wireless interface to a digital processor 1810 of an electronic control circuit 1812 (similar to the electronic control assembly 1336 of the previous embodiment in terms of its processing of data in accordance with the testing algorithm of this disclosure). The wireless RF transmitter device 1804 is configured to transmit data readings of the gas sensor assembly 1814 to the RF wireless receiver device 1806. Transmission is performed under the control of the digital processor 1816.

It will be understood that the RF wireless receiver 1806 replaces the card edge connector assembly of the previous embodiment for receiving data regarding gas sensor readings from the gas monitor 1802. The RF transmitter device 1804 replaces the signal contacts (not shown) on the printed circuit board (not shown) of the previous embodiment for transmitting the data. The wireless RF transmitter device 1804 is connected through an interface to an electronic control assembly 1836 of the gas monitor 1802. The electronic control assembly 1836 is similar to the electronic control assembly of the gas monitor of the previous embodiment in terms of its function in transmitting the test data of the gas sensor. The digital processor 1816 of the electronic control assembly 1836 may instruct the gas sensor assembly to operate at discrete time intervals or relatively continuously so as to take sensor readings during testing and transmit these readings to the digital processor 1810 of the electronic control assembly 1812 of the wireless testing tool 1800. The transmitted data is digital. Exemplary RF protocols may be used and these include, but are not limited to Bluetooth™, Zigbee™, 802.11a/b/g, and CC1000. The distances the wireless information is transmitted can be controlled in a known fashion. While this embodiment describes a one-way system, it will be noted that bidirectional transmission may be implemented as well. In this latter regard, a wireless transceiver would be used in both the wireless testing tool 1800 and the gas monitor. Such an approach may be used in a computer network as described below wherein the wireless approach would rely upon suitable wireless protocols for information transmission.

The overall operation of the portable testing tool 1800 is different in how the data is transmitted and received. Of course, with wireless, the housing assembly of the portable device need not be provided with mating recesses to assist in properly aligning the testing tool to the gas monitor in order to transmit data. As noted, other suitable wireless approaches may be used, such as infrared (IR), visible or acoustic energy. In regard to IR, the gas monitor would have its electronic control assembly of the data transmitting unit provided with a photodiode that cooperates with a photodetectors or photosensors of the testing tool 1800. Other than the mode of wireless transmissions, the electronic control assembly 1812 of the testing tool operates as describe above in regards to the previous embodiment insofar as it includes the testing module application for handling the data according to the testing algorithm noted. Accordingly, the process of operating the testing tool 1800 is the same as in the previous embodiments in terms of responding to the readings of the gas sensor during the testing mode. In this regard, the housing assembly is provided with similar Test and Start switches 1821a and 1821b; respectively, that operate as the switches (1321a and 1331b) of the previous embodiment in terms of commencing different aspects of the method.

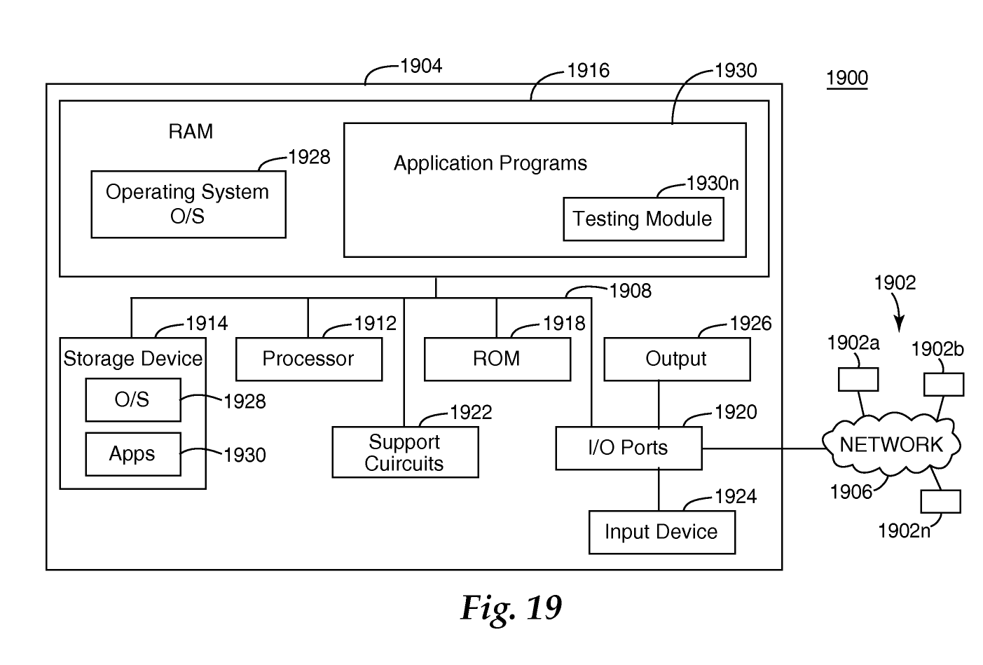

Reference is now made to FIG. 19 for illustrating an exemplary embodiment of a gas testing system 1900 that may be used for evaluating the performance of gas monitor 1902a-n (collectively, 1902) that may be linked to a programmable electronic system 1904 through a computer network 1906. The network may be any one of several suitable types through which data may be transferred. For instance, the computer network 1906 can be a wireless network as in the present embodiment. Other typical types of networks may include local-area network (LAN), wide area network (WAN), or for that matter the internet The programmable electronic system 1904 may represent any type of programmable electronic device, such as computer system 1904, programmable logic devices, or the like. The computer system 1904 may include portable computer systems including laptops, handheld computer systems. Other computer systems include client computers, servers, PC-based servers, minicomputers, midrange computers, mainframe computers; or other suitable devices.

In one exemplary embodiment, the computer system 1904 is a commercially available laptop computer system 1904. The laptop computer system 1904 includes an interconnect bus 1908. Various components of the computer system are coupled and communicate with each other through the interconnect bus. Coupled to the system interconnect bus 1908 is at least a single processor unit 1912, a storage unit, such as a random access memory (RAM) 1916, read only memory (ROM) 1918, input/output (I/O) ports 1920 and other support circuits 1922 that include controllers for the graphics display, or the like (not shown). The input and output devices 1924 and 1926; respectively, permit user interaction with the computer system 1904. The input/output ports 1920 can include various controllers (not shown) for each of the input devices 1924, such as a keyboard 1924 (FIG. 19), mouse, joystick user interface, or the like. As a result, the gas monitor to be tested can be selected from a computer monitored group of gas monitors. The I/O ports 1920 may be suitably connected to the network 1906 as through an Ethernet cable or the like. In this embodiment, there is provided a wireless RF transceiver network interface that interfaces with the processor and memory to permit the wireless communication with a remote gas monitor including a suitable transceiver as noted.

The processor unit 1912 sends and receives instructions and data information to and from each of the computer systems' components that are coupled to the interconnect bus so as to perform system operations based upon the requirements of the computer system's operating system (OS) 1928 and other specialized applications 1930a-n (collectively referred to as application programs 1930). One of the specialized programs 1930 is a testing module application 1930n that contains aspects of the testing module applications noted above that enable it to perform as noted above to achieve a validation determination. The code stored in the ROM 1918 typically controls the basic hardware operations. Those skilled in the art will appreciate that the testing mode module is capable of being distributed as a computer program product in a variety of forms, such as tangible media that can be processed by a processor, and that the disclosure applies equally regardless of the particular type of signal bearing media used to actually carry out the distribution. The storage device 1914 can be a permanent storage medium, such as a hard disk, CD ROM, tape, or the like which stores the operating system 1928 and the specialized application programs 1930. The program code of the operating system(s) and/or the applications program 1930n is sent to the RAM 1916 for temporary storage and subsequent execution by the processor unit 1912. The contents of the RAM 1916 may be retrieved from the storage device 1914 as required. Illustratively, the RAM 1916 is shown with the operating system 1928 and application programs 1930 concurrently stored therein.

The testing module application 1930n operates as noted in the operation of the portable testing tool described in FIG. 19. Thus, the sequence of steps carried out in the process of this embodiment are essentially the same as with those described above in regard to the FIG. 17, except instead of a switch button being actuated, the input device 1924 is appropriately actuated. Hence, in the network system 1906, the initialization process may occur in response to a user activating the input device 1924 of the laptop computer system so as to wirelessly be coupled to one or more gas monitors. The testing module application 1930n will identify all the linked gas monitors 1902. Thereafter, a user or tester may select one of the identified gas monitors to be tested through the input device 1924 to the laptop computer. Once a gas monitor to be tested is selected, the testing process 1700 of the present disclosure is commenced. Thereafter, the selected gas monitor is instructed to capture a reading of the ambient air surrounding its gas sensor. Thereafter, the testing module application 1930n makes a determination as to whether or not the reading is captured successfully. In this regard, if a trouble fault condition is determined, such result may be displayed on the output display 1926, thereby alerting the tester or user of the ambient air conditions which exist at the gas monitor being tested. Such an alert may be displayed on the output device 1926, such as a monitor.

The testing module application 1930n operates in the sequence carried out in the blocks 1714-1740, as noted above. As a result, the testing module application 1930n performs a process that allows for an accelerated processing of the test data for determining if a passing condition of the gas sensor assembly has been reached with the gas sensor assembly being operated in a normal mode. In determining if a passing condition has been reached, the testing module includes: obtaining a first reading value of testing gas applied to the gas sensor assembly, storing the first reading value, obtaining a second gas sensor assembly reading value, determining a rate-of-rise value of the first and second reading values based on a difference of the first and second reading values relative to a testing time interval therebetween, and, determining if a gas sensor assembly passing condition exists based on a comparison of the rate-of-rise value to at least a first predefined rate-of-rise value of the gas sensor assembly after testing gas is applied. Further, the determining process includes determining if the passing condition exists if the rate-of-rise value of the first and second reading values is greater than a second predefined rate-of-rise value of the gas sensor assembly after testing gas is applied.

The present disclosure also contemplates a gas monitor field testing kit 2000 (FIGS. 13 and 14). In one illustrated embodiment, the gas monitor field testing kit 2000 includes the fluid coupler 1306 and the portable gas testing system 1300 which are particularly adapted for use in combination with the gas monitor assembly 1302. As such, a highly versatile approach is provided for testing a wide variety of gas monitors. As noted, the portable field testing kit 2000 is also couplable to a computer network. In the field testing kit 2000 provision is made for a source of testing gas, such as of the kind noted above. While the field testing kit prefers use of the noted fluid coupler 1306, it will be understood that a wide variety of other fluid couplers may be used in this regard.

Aspects of the disclosure also include a method and system utilized for validating gas monitor performance in a manner that reduces testing gas and labor costs. Further aspects of the disclosure include improving upon validating gas monitor performance by achieving the above in a manner that enables portability of the testing process. Further aspects of the disclosure include improving upon known methods and systems utilized for testing the performance of gas monitors through implementation of a handheld testing device. Still further aspects of the disclosure include improving upon known methods and systems utilized by quickly testing the performance of gas monitors thorough a portable testing device couplable to a network. Still further aspects of the disclosure include improving upon known methods and systems, wherein testing procedures are performed in an even more economical and expeditious manner by using a portable gas monitor testing device having a testing module onboard instead of being incorporated into each monitor to be tested. Other aspects of the disclosure include improving upon known methods and systems, wherein provision is made for a gas testing kit that includes both a fluid coupling device and a gas testing system that facilitate a highly versatile testing procedure. Aspects of the disclosure include methods and systems for significantly reducing the actual testing time of testing gas monitors. Aspects also include allowing an accelerated processing of the test data for determining if a passing condition of the gas sensor assembly has been reached with the gas sensor assembly being operated in a normal mode. The aspects described herein are merely a few of the several that can be achieved by using the disclosure. The foregoing descriptions thereof do not suggest that the disclosure must only be utilized in a specific manner to attain the foregoing aspects.

The above embodiments have been described as being accomplished in a particular sequence, it will be appreciated that such sequences of the operations may change and still remain within the scope of the disclosure. For example, an illustrated embodiment discusses one set of testing protocols wherein the minimum validation value for the gas monitor must be satisfied before applying testing gas to obtain a first reading. It will be appreciated that such preliminary procedures need not be followed for one to conduct testing of gas sensor assemblies. Also, other procedures may be added.

This disclosure may take on various modifications and alterations without departing from the spirit and scope. Accordingly, this disclosure is not limited to the above-described embodiments, but is to be controlled by limitations set forth in the following claims and any equivalents thereof

What is claimed is:

1. A method adapted for testing a gas sensor assembly, the method comprises: coupling a portable tool to the gas sensor assembly; receiving test data representative of performances of the gas sensor assembly in response to testing gas by a data receiving device on the portable tool; and determining performance of the gas sensor assembly based on processing the received test data.

2. The method of claim 1, further comprising coupling the data receiving device to a data transmitting device of the gas sensor assembly which transmits the test data.

3. The method of claim 1, further including preliminarily determining if a minimum level of response of the gas sensor assembly is reached before applying testing gas to the gas sensor assembly.

4. The method of claim 3, wherein testing gas is applied to the gas sensor assembly in response to the minimum level being reached.

5. The method of claim 4, wherein the testing gas is applied by a fluid coupling apparatus that is fluidly couplable to the gas sensor assembly.

6. The method of claim 2, wherein the data receiving device is physically coupled to the data transmitting device of the gas sensor assembly.

7. The method of claim 2, wherein the data receiving device is wirelessly coupled to a data transmitting device of the gas sensor assembly.

8. The method of claim 2, wherein the data receiving device is coupled to the data transmitting device of the gas sensor assembly through a network.

9. The method of claim 7, wherein the wireless coupling includes coupling by RF.

10. The method of claim 1, wherein the determining performance of the gas sensor assembly includes utilizing a testing module that allows an accelerated processing of the test data for determining if a passing condition of the gas sensor assembly has been reached with the gas sensor assembly being operated in a normal mode.

11. The method of claim 10, wherein the determining performance of the gas sensor assembly includes: obtaining a first reading value of testing gas applied to the gas sensor assembly, storing the first reading value, obtaining a second gas sensor assembly reading value, determining a rate-of-rise value of the first and second reading values based on a difference of the first and second reading values relative to a testing time interval therebetween, and determining if a gas sensor assembly passing condition exists based on a comparison of the rate-of-rise value to at least a first predefined rate-of-rise value of the gas sensor assembly.

12. The method of claim 11, wherein the determining includes determining if the passing condition exists if the rate-of-rise value of the first and second reading values is greater than a second predefined rate-of-rise value of the gas sensor assembly.

13. The method of claim 10, wherein the coupling of the portable tool includes coupling a portable computer system that performs the data receiving and the determining.

14. A gas testing system adapted for use in testing a gas sensor assembly, the system comprising: a portable tool; a data receiving device on the portable tool; a data processing system on the portable tool and including a testing module; the data processing system is operable for receiving test data relating to testing gas values sensed by a gas sensor assembly, wherein the testing module is operable for determining performance of the gas sensor assembly based on the received test data.

15. The gas testing system of claim 14, wherein the testing module preliminarily determines if a minimum level of response of the gas sensor assembly is reached before allowing testing gas to be applied to the gas sensor assembly.

16. The gas testing system of claim 14, wherein the data receiving device includes a connector assembly for physically coupling to contacts on the gas sensor assembly.

17. The gas testing system of claim 14, wherein the data receiving device includes a wireless receiver for wirelessly coupling to a transmitting device of the gas sensor assembly.

18. The gas testing system of claim 17, wherein the wireless receiver utilizes RF.

19. The gas testing system of claim 14, wherein the portable tool, the data processing system, and the data receiving device are included in a portable computer system.

20. The gas testing system of claim 14, wherein the data processing system includes a digital processor, a memory coupled to the digital processor and operated on by the digital processor, and the testing module resides in the memory.

21. The gas testing system of claim 14, wherein the testing module allows an accelerated processing of the test data for determining if a passing condition of the gas sensor assembly has been reached with the gas sensor assembly being operated in a normal mode.

22. The gas testing system of claim 2 1, wherein the testing module obtains a first reading value of testing gas applied to the gas sensor assembly, stores the first reading value, obtains a second gas sensor assembly reading value, determines a rate-of-rise value of the first and second reading values based on a difference of the first and second reading values relative to a testing time interval therebetween, and, determines if a gas sensor assembly passing condition exists based on a comparison of the rate-of-rise value to at least a first predefined rate-of-rise value of the gas sensor assembly.

23. The gas testing system of claim 22, wherein the testing module determines if the passing condition exists if the rate-of-rise value of the first and second reading values is greater than a second predefined rate-of-rise value of the gas sensor assembly.

24. The gas testing system of claim 14, wherein the data receiving device is couplable to a data transmitting assembly of a gas sensor assembly through a network.

25. A gas monitor testing kit comprising: a portable gas testing system for testing performance of a gas sensor assembly adapted for use in a gas monitor assembly that is subjected to testing gas; and a fluid coupling apparatus for delivering testing gas to a gas sensor assembly of a gas monitor assembly.

26. The kit of claim 25, further including a source of testing gas.

27. The kit of claim 25, wherein the portable gas testing system comprises: a portable housing; a data receiving device carried by the portable housing; a data processing system on the portable tool and including a testing module; the data processing system being operable for receiving test data relating to testing gas values sensed by a gas sensor assembly, wherein the testing module is operable for determining performance of a gas sensor assembly based on the received test data.

28. The kit of claim 25, wherein the fluid coupling apparatus comprises: a fluid coupler removably couplable to a gas monitor assembly so as to be positioned interior of a gas monitor housing of a gas monitor assembly; and, a fluid passageway carried by the coupler and including a gas delivery opening for delivering testing gas directly to a gas sensor assembly interior of a gas monitor assembly when coupled thereto.

29. The kit of claim 28, wherein the gas delivery opening is generally aligned with a gas sensor assembly when coupled.

30. The kit of claim 25, wherein the gas testing system includes a connector assembly for physically coupling to contacts on a gas sensor assembly.

31. The kit of claim 25, wherein the gas testing system includes a wireless receiver assembly for wirelessly coupling to a wireless transmitting device of a gas sensor assembly.

32. The kit of claim 25, wherein the wireless receiver assembly couples utilizing RF.

33. The kit of claim 25, wherein the portable gas testing system is a portable computer system.

34. The kit of claim 27, wherein the testing module allows an accelerated processing of the test data for determining if a passing condition of a gas sensor assembly has been reached with a gas sensor assembly being operated in a normal mode.

35. The kit of claim 34, wherein the testing module obtains a first reading value of testing gas applied to a gas sensor assembly, stores the first reading value, obtains a second gas sensor assembly reading value, determines a rate-of-rise value of the first and second reading values based on a difference of the first and second reading values relative to a testing time interval therebetween, and, determines if a gas sensor assembly passing condition exists based on a comparison of the rate-of-rise value to at least a first predefined rate-of-rise value of a gas sensor assembly.

36. The kit of claim 35, wherein testing module determines if the passing condition exists if the rate-of-rise value of the first and second reading values is greater than a second predefined rate-of-rise value of the gas sensor assembly.

37. The kit of claim 25, wherein the data receiving device is couplable to a data transmitting device of a gas sensor assembly through a network.
